SEQUENCE LISTING (141 sequences)

SEQ ID NO: 1 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 2 - protein, 255 aa, Homo sapiens
SLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 255 aa, Artificial Sequence
  Synthetic Polypeptide
SLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 255 aa, Artificial Sequence
  Synthetic Polypeptide
SLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 255 aa, Artificial Sequence
  Synthetic Polypeptide
SLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 255 aa, Artificial Sequence
  Synthetic Polypeptide
SLGTQTYICNVNHKPSNTKVDKKVEPKSCDKTHTCPPCPAPELLGGPSVFLFPPKPKDTLMISRTPEVTCVVVDVSFEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 255 aa, Artificial Sequence
  Synthetic Polypeptide
SLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 255 aa, Artificial Sequence
  Synthetic Polypeptide
SLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 255 aa, Artificial Sequence
  Synthetic Polypeptide
SLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 255 aa, Artificial Sequence
  Synthetic Polypeptide
SLGTQTYICNVNHKPSNTKVDKKVEPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 255 aa, Artificial Sequence
  Synthetic Polypeptide
SLGTQTYICNVNHKPSNTKVDKKVEPKSCDKTHTCPPCPAPELLGGPDVFLFPPKPKDTLMISRTPEVTCVVVDVSFEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 8 aa, Artificial Sequence
  Synthetic Polypeptide
GFTFTSYA

SEQ ID NO: 13 - protein, 8 aa, Artificial Sequence
  Synthetic Polypeptide
ISHGGTYT

SEQ ID NO: 14 - protein, 16 aa, Artificial Sequence
  Synthetic Polypeptide
ARHPIYSGNYQGYFDY

SEQ ID NO: 15 - protein, 5 aa, Artificial Sequence
  Synthetic Polypeptide
SGVNY

SEQ ID NO: 16 - protein, 3 aa, Artificial Sequence
  Synthetic Polypeptide
FTS

SEQ ID NO: 17 - protein, 9 aa, Artificial Sequence
  Synthetic Polypeptide
QQFTSSPYT

SEQ ID NO: 18 - protein, 123 aa, Artificial Sequence
  Synthetic Polypeptide
EVQLLESGGGLVQPGGSLRLSCAASGFTFTSYAMSWVRQAPGKGLEWVAVISHGGTYTYYADSVKGRFTISRDNAKNTLYLQMNSLRAEDTAVYYCARHPIYSGNYQGYFDYWGQGTLVTVSS

SEQ ID NO: 19 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAMYYCARHPIYSGNYQGYFDYWGQGTLVTVSS

SEQ ID NO: 20 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGNYQGYFDYWGQGTLVTVSS

SEQ ID NO: 21 - protein, 106 aa, Artificial Sequence
  Synthetic Polypeptide
DIQLTQSPSFLSASVGDRVTITCRASSGVNYLYWYQQKPGKAPKLWIYFTSTLAPGVPSRFSGSGSGNEYTLTISSLQPEDFATYYCQQFTSSPYTFGGGTKLEIK

SEQ ID NO: 22 - protein, 106 aa, Artificial Sequence
  Synthetic Polypeptide
DIQLTQSPSFLSASVGDRVTITCRASSGVNYLYWYQQKPGKAPKLWIYFTSTLAPGVPSRFSGSGSGNEYTLTISSLQPEDFATYYCQQFTSSPYTFGQGTKLEIK

SEQ ID NO: 23 - protein, 106 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFTSSPYTFGGGTKLEIK

SEQ ID NO: 24 - protein, 106 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFTSSPYTFGQGTKLEIK

SEQ ID NO: 25 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAMYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAMYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAMYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 452 aa, Artificial Sequence
  Synthetic Polypeptide
EVQLLESGGGLVQPGGSLRLSCAASGFTFTSYAMSWVRQAPGKGLEWVAVISHGGTYTYYADSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 452 aa, Artificial Sequence
  Synthetic Polypeptide
EVQLLESGGGLVQPGGSLRLSCAASGFTFTSYAMSWVRQAPGKGLEWVAVISHGGTYTYYADSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 452 aa, Artificial Sequence
  Synthetic Polypeptide
EVQLLESGGGLVQPGGSLRLSCAASGFTFTSYAMSWVRQAPGKGLEWVAVISHGGTYTYYADSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSFEDPEVKFNWYVDGVEVHNAKTKPREEQYNSTYRVVSVLTVLHQDWLNGKEYKCKVTNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 8 aa, Artificial Sequence
  Synthetic Polypeptide
GFTFRSYA

SEQ ID NO: 35 - protein, 8 aa, Artificial Sequence
  Synthetic Polypeptide
ISHKGTYT

SEQ ID NO: 36 - protein, 8 aa, Artificial Sequence
  Synthetic Polypeptide
ISHKGKYT

SEQ ID NO: 37 - protein, 8 aa, Artificial Sequence
  Synthetic Polypeptide
ISHRGTYT

SEQ ID NO: 38 - protein, 8 aa, Artificial Sequence
  Synthetic Polypeptide
ISHRGRYT

SEQ ID NO: 39 - protein, 8 aa, Artificial Sequence
  Synthetic Polypeptide
ISRGGTYT

SEQ ID NO: 40 - protein, 8 aa, Artificial Sequence
  Synthetic Polypeptide
ISSGGTYT

SEQ ID NO: 41 - protein, 8 aa, Artificial Sequence
  Synthetic Polypeptide
ISHGGKYT

SEQ ID NO: 42 - protein, 8 aa, Artificial Sequence
  Synthetic Polypeptide
ISHGGRYT

SEQ ID NO: 43 - protein, 8 aa, Artificial Sequence
  Synthetic Polypeptide
ISHGGWYT

SEQ ID NO: 44 - protein, 8 aa, Artificial Sequence
  Synthetic Polypeptide
ISHGGYYT

SEQ ID NO: 45 - protein, 16 aa, Artificial Sequence
  Synthetic Polypeptide
ARHPIYSGFYQGYFDY

SEQ ID NO: 46 - protein, 16 aa, Artificial Sequence
  Synthetic Polypeptide
ARHPIYSGWYQGYFDY

SEQ ID NO: 47 - protein, 16 aa, Artificial Sequence
  Synthetic Polypeptide
ARHPIYSGQYQGYFDY

SEQ ID NO: 48 - protein, 16 aa, Artificial Sequence
  Synthetic Polypeptide
ARHPIYAGNYQGYFDY

SEQ ID NO: 49 - protein, 16 aa, Artificial Sequence
  Synthetic Polypeptide
ARHPIYCGNYQGYFDY

SEQ ID NO: 50 - protein, 16 aa, Artificial Sequence
  Synthetic Polypeptide
ARHPIYDGNYQGYFDY

SEQ ID NO: 51 - protein, 16 aa, Artificial Sequence
  Synthetic Polypeptide
ARHPIYEGNYQGYFDY

SEQ ID NO: 52 - protein, 16 aa, Artificial Sequence
  Synthetic Polypeptide
ARHPIYFGNYQGYFDY

SEQ ID NO: 53 - protein, 16 aa, Artificial Sequence
  Synthetic Polypeptide
ARHPIYGGNYQGYFDY

SEQ ID NO: 54 - protein, 16 aa, Artificial Sequence
  Synthetic Polypeptide
ARHPIYHGNYQGYFDY

SEQ ID NO: 55 - protein, 16 aa, Artificial Sequence
  Synthetic Polypeptide
ARHPIYIGNYQGYFDY

SEQ ID NO: 56 - protein, 16 aa, Artificial Sequence
  Synthetic Polypeptide
ARHPIYKGNYQGYFDY

SEQ ID NO: 57 - protein, 16 aa, Artificial Sequence
  Synthetic Polypeptide
ARHPIYLGNYQGYFDY

SEQ ID NO: 58 - protein, 16 aa, Artificial Sequence
  Synthetic Polypeptide
ARHPIYMGNYQGYFDY

SEQ ID NO: 59 - protein, 16 aa, Artificial Sequence
  Synthetic Polypeptide
ARHPIYNGNYQGYFDY

SEQ ID NO: 60 - protein, 16 aa, Artificial Sequence
  Synthetic Polypeptide
ARHPIYPGNYQGYFDY

SEQ ID NO: 61 - protein, 16 aa, Artificial Sequence
  Synthetic Polypeptide
ARHPIYQGNYQGYFDY

SEQ ID NO: 62 - protein, 16 aa, Artificial Sequence
  Synthetic Polypeptide
ARHPIYRGNYQGYFDY

SEQ ID NO: 63 - protein, 16 aa, Artificial Sequence
  Synthetic Polypeptide
ARHPIYTGNYQGYFDY

SEQ ID NO: 64 - protein, 16 aa, Artificial Sequence
  Synthetic Polypeptide
ARHPIYVGNYQGYFDY

SEQ ID NO: 65 - protein, 16 aa, Artificial Sequence
  Synthetic Polypeptide
ARHPIYWGNYQGYFDY

SEQ ID NO: 66 - protein, 16 aa, Artificial Sequence
  Synthetic Polypeptide
ARHPIYYGNYQGYFDY

SEQ ID NO: 67 - protein, 8 aa, Artificial Sequence
  Synthetic Polypeptide
ISHGGRYT

SEQ ID NO: 68 - protein, 16 aa, Artificial Sequence
  Synthetic Polypeptide
ARHPIHSGNYQGYFDY

SEQ ID NO: 69 - protein, 5 aa, Artificial Sequence
  Synthetic Polypeptide
SGVIY

SEQ ID NO: 70 - protein, 5 aa, Artificial Sequence
  Synthetic Polypeptide
SGVKY

SEQ ID NO: 71 - protein, 5 aa, Artificial Sequence
  Synthetic Polypeptide
SGVRY

SEQ ID NO: 72 - protein, 5 aa, Artificial Sequence
  Synthetic Polypeptide
SGVSY

SEQ ID NO: 73 - protein, 5 aa, Artificial Sequence
  Synthetic Polypeptide
SGVWY

SEQ ID NO: 74 - protein, 5 aa, Artificial Sequence
  Synthetic Polypeptide
SGVYY

SEQ ID NO: 75 - protein, 9 aa, Artificial Sequence
  Synthetic Polypeptide
QQFTRSPYT

SEQ ID NO: 76 - protein, 9 aa, Artificial Sequence
  Synthetic Polypeptide
QQFTSKPYT

SEQ ID NO: 77 - protein, 9 aa, Artificial Sequence
  Synthetic Polypeptide
QQFKSSPYT

SEQ ID NO: 78 - protein, 9 aa, Artificial Sequence
  Synthetic Polypeptide
QQFRSSPYT

SEQ ID NO: 79 - protein, 9 aa, Artificial Sequence
  Synthetic Polypeptide
QQFYSSPYT

SEQ ID NO: 80 - protein, 123 aa, Artificial Sequence
  Synthetic Polypeptide
EVQLLESGGGLVQPGGSLRLSCAASGFTFTSYAMSWVRQAPGKGLEWVATISHKGTYTYYPDSVKGRFTISRDNAKNTLYLQMNSLRAEDTAVYYCARHPIYSGNYQGYFDYWGQGTLVTVSS

SEQ ID NO: 81 - protein, 123 aa, Artificial Sequence
  Synthetic Polypeptide
EVQLLESGGGLVQPGGSLRLSCAASGFTFTSYAMSWVRQAPGKGLEWVATISHKGTYTYYPDSVKGRFTISRDNAKNTLYLQMNSLRAEDTAVYYCARHPIYSGFYQGYFDYWGQGTLVTVSS

SEQ ID NO: 82 - protein, 123 aa, Artificial Sequence
  Synthetic Polypeptide
EVQLLESGGGLVQPGGSLRLSCAASGFTFTSYAMSWVRQAPGKGLEWVATISHKGKYTYYPDSVKGRFTISRDNAKNTLYLQMNSLRAEDTAVYYCARHPIYSGFYQGYFDYWGQGTLVTVSS

SEQ ID NO: 83 - protein, 123 aa, Artificial Sequence
  Synthetic Polypeptide
EVQLLESGGGLVQPGGSLRLSCAASGFTFTSYAMSWVRQAPGKGLEWVATISHRGTYTYYPDSVKGRFTISRDNAKNTLYLQMNSLRAEDTAVYYCARHPIYSGNYQGYFDYWGQGTLVTVSS

SEQ ID NO: 84 - protein, 123 aa, Artificial Sequence
  Synthetic Polypeptide
EVQLLESGGGLVQPGGSLRLSCAASGFTFTSYAMSWVRQAPGKGLEWVATISHRGTYTYYPDSVKGRFTISRDNAKNTLYLQMNSLRAEDTAVYYCARHPIYSGFYQGYFDYWGQGTLVTVSS

SEQ ID NO: 85 - protein, 123 aa, Artificial Sequence
  Synthetic Polypeptide
EVQLLESGGGLVQPGGSLRLSCAASGFTFTSYAMSWVRQAPGKGLEWVATISHRGTYTYYPDSVKGRFTISRDNAKNTLYLQMNSLRAEDTAVYYCARHPIYSGWYQGYFDYWGQGTLVTVSS

SEQ ID NO: 86 - protein, 123 aa, Artificial Sequence
  Synthetic Polypeptide
EVQLLESGGGLVQPGGSLRLSCAASGFTFTSYAMSWVRQAPGKGLEWVATISHRGRYTYYPDSVKGRFTISRDNAKNTLYLQMNSLRAEDTAVYYCARHPIYSGNYQGYFDYWGQGTLVTVSS

SEQ ID NO: 87 - protein, 123 aa, Artificial Sequence
  Synthetic Polypeptide
EVQLLESGGGLVQPGGSLRLSCAASGFTFTSYAMSWVRQAPGKGLEWVATISHRGRYTYYPDSVKGRFTISRDNAKNTLYLQMNSLRAEDTAVYYCARHPIYSGFYQGYFDYWGQGTLVTVSS

SEQ ID NO: 88 - protein, 123 aa, Artificial Sequence
  Synthetic Polypeptide
EVQLLESGGGLVQPGGSLRLSCAASGFTFTSYAMSWVRQAPGKGLEWVATISRGGTYTYYPDSVKGRFTISRDNAKNTLYLQMNSLRAEDTAVYYCARHPIYSGNYQGYFDYWGQGTLVTVSS

SEQ ID NO: 89 - protein, 123 aa, Artificial Sequence
  Synthetic Polypeptide
EVQLLESGGGLVQPGGSLRLSCAASGFTFTSYAMSWVRQAPGKGLEWVATISRGGTYTYYPDSVKGRFTISRDNAKNTLYLQMNSLRAEDTAVYYCARHPIYSGQYQGYFDYWGQGTLVTVSS

SEQ ID NO: 90 - protein, 123 aa, Artificial Sequence
  Synthetic Polypeptide
EVQLLESGGGLVQPGGSLRLSCAASGFTFTSYAMSWVRQAPGKGLEWVATISSGGTYTYYPDSVKGRFTISRDNAKNTLYLQMNSLRAEDTAVYYCARHPIYSGNYQGYFDYWGQGTLVTVSS

SEQ ID NO: 91 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGFYQGYFDYWGQGTLVTVSS

SEQ ID NO: 92 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGWYQGYFDYWGQGTLVTVSS

SEQ ID NO: 93 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYAGNYQGYFDYWGQGTLVTVSS

SEQ ID NO: 94 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYCGNYQGYFDYWGQGTLVTVSS

SEQ ID NO: 95 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYDGNYQGYFDYWGQGTLVTVSS

SEQ ID NO: 96 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYEGNYQGYFDYWGQGTLVTVSS

SEQ ID NO: 97 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYFGNYQGYFDYWGQGTLVTVSS

SEQ ID NO: 98 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYGGNYQGYFDYWGQGTLVTVSS

SEQ ID NO: 99 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYHGNYQGYFDYWGQGTLVTVSS

SEQ ID NO: 100 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYIGNYQGYFDYWGQGTLVTVSS

SEQ ID NO: 101 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYKGNYQGYFDYWGQGTLVTVSS

SEQ ID NO: 102 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYLGNYQGYFDYWGQGTLVTVSS

SEQ ID NO: 103 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYMGNYQGYFDYWGQGTLVTVSS

SEQ ID NO: 104 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYNGNYQGYFDYWGQGTLVTVSS

SEQ ID NO: 105 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYPGNYQGYFDYWGQGTLVTVSS

SEQ ID NO: 106 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYQGNYQGYFDYWGQGTLVTVSS

SEQ ID NO: 107 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYRGNYQGYFDYWGQGTLVTVSS

SEQ ID NO: 108 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYTGNYQGYFDYWGQGTLVTVSS

SEQ ID NO: 109 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYVGNYQGYFDYWGQGTLVTVSS

SEQ ID NO: 110 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYWGNYQGYFDYWGQGTLVTVSS

SEQ ID NO: 111 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYYGNYQGYFDYWGQGTLVTVSS

SEQ ID NO: 112 - protein, 123 aa, Artificial Sequence
  Synthetic Polypeptide
EVQLLESGGGLVQPGGSLRLSCAASGFTFRSYAMSWVRQAPGKGLEWVATISHGGTYTYYPDSVKGRFTISRDNAKNTLYLQMNSLRAEDTAVYYCARHPIYSGNYQGYFDYWGQGTLVTVSS

SEQ ID NO: 113 - protein, 123 aa, Artificial Sequence
  Synthetic Polypeptide
EVQLLESGGGLVQPGGSLRLSCAASGFTFTSYAMSWVRQAPGKGLEWVATISHGGKYTYYPDSVKGRFTISRDNAKNTLYLQMNSLRAEDTAVYYCARHPIYSGNYQGYFDYWGQGTLVTVSS

SEQ ID NO: 114 - protein, 123 aa, Artificial Sequence
  Synthetic Polypeptide
EVQLLESGGGLVQPGGSLRLSCAASGFTFTSYAMSWVRQAPGKGLEWVATISHGGKYTYYPDSVKGRFTISRDNAKNTLYLQMNSLRAEDTAVYYCARHPIYSGWYQGYFDYWGQGTLVTVSS

SEQ ID NO: 115 - protein, 123 aa, Artificial Sequence
  Synthetic Polypeptide
EVQLLESGGGLVQPGGSLRLSCAASGFTFTSYAMSWVRQAPGKGLEWVATISHGGRYTYYPDSVKGRFTISRDNAKNTLYLQMNSLRAEDTAVYYCARHPIYSGNYQGYFDYWGQGTLVTVSS

SEQ ID NO: 116 - protein, 123 aa, Artificial Sequence
  Synthetic Polypeptide
EVQLLESGGGLVQPGGSLRLSCAASGFTFTSYAMSWVRQAPGKGLEWVATISHGGRYTYYPDSVKGRFTISRDNAKNTLYLQMNSLRAEDTAVYYCARHPIYSGFYQGYFDYWGQGTLVTVSS

SEQ ID NO: 117 - protein, 123 aa, Artificial Sequence
  Synthetic Polypeptide
EVQLLESGGGLVQPGGSLRLSCAASGFTFTSYAMSWVRQAPGKGLEWVATISHGGWYTYYPDSVKGRFTISRDNAKNTLYLQMNSLRAEDTAVYYCARHPIYSGNYQGYFDYWGQGTLVTVSS

SEQ ID NO: 118 - protein, 123 aa, Artificial Sequence
  Synthetic Polypeptide
EVQLLESGGGLVQPGGSLRLSCAASGFTFTSYAMSWVRQAPGKGLEWVATISHGGYYTYYPDSVKGRFTISRDNAKNTLYLQMNSLRAEDTAVYYCARHPIYSGNYQGYFDYWGQGTLVTVSS

SEQ ID NO: 119 - protein, 123 aa, Artificial Sequence
  Synthetic Polypeptide
EVQLLESGGGLVQPGGSLRLSCAASGFTFTSYAMSWVRQAPGKGLEWVATISHGGTKTYYPDSVKGRFTISRDNAKNTLYLQMNSLRAEDTAVYYCARHPIYSGNYQGYFDYWGQGTLVTVSS

SEQ ID NO: 120 - protein, 123 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIHSGNYQGYFDYWGQGTLVTVSS

SEQ ID NO: 121 - protein, 123 aa, Artificial Sequence
  Synthetic Polypeptide
EVQLLESGGGLVQPGGSLRLSCAASGFTFTSYAMSWVRQAPGKGLEWVARISHGGTYTYYPDSVKGRFTISRDNAKNTLYLQMNSLRAEDTAVYYCARHPIYSGNYQGYFDYWGQGTLVTVSS

SEQ ID NO: 122 - protein, 123 aa, Artificial Sequence
  Synthetic Polypeptide
EVQLLESGGGLVQPGGSLRLSCAASGFTFTSYAMSWVRQAPGKGLEWVATISHGGTYTWYPDSVKGRFTISRDNAKNTLYLQMNSLRAEDTAVYYCARHPIYSGNYQGYFDYWGQGTLVTVSS

SEQ ID NO: 123 - protein, 106 aa, Artificial Sequence
  Synthetic Polypeptide
DIQLTQSPSFLSASVGDRVTITCRASSGVIYLYWYQQKPGKAPKLWIYFTSTLASGVPSRFSGSGSGNEYTLTISSLQPEDFATYYCQQFTSSPYTFGQGTKLEIK

SEQ ID NO: 124 - protein, 106 aa, Artificial Sequence
  Synthetic Polypeptide
DIQLTQSPSFLSASVGDRVTITCRASSGVKYLYWYQQKPGKAPKLWIYFTSTLASGVPSRFSGSGSGNEYTLTISSLQPEDFATYYCQQFTSSPYTFGQGTKLEIK

SEQ ID NO: 125 - protein, 106 aa, Artificial Sequence
  Synthetic Polypeptide
DIQLTQSPSFLSASVGDRVTITCRASSGVRYLYWYQQKPGKAPKLWIYFTSTLASGVPSRFSGSGSGNEYTLTISSLQPEDFATYYCQQFTSSPYTFGQGTKLEIK

SEQ ID NO: 126 - protein, 106 aa, Artificial Sequence
  Synthetic Polypeptide
DIQLTQSPSFLSASVGDRVTITCRASSGVRYLYWYQQKPGKAPKLWIYFTSTLASGVPSRFSGSGSGNEYTLTISSLQPEDFATYYCQQFTRSPYTFGQGTKLEIK

SEQ ID NO: 127 - protein, 106 aa, Artificial Sequence
  Synthetic Polypeptide
DIQLTQSPSFLSASVGDRVTITCRASSGVSYLYWYQQKPGKAPKLWIYFTSTLASGVPSRFSGSGSGNEYTLTISSLQPEDFATYYCQQFTSSPYTFGQGTKLEIK

SEQ ID NO: 128 - protein, 106 aa, Artificial Sequence
  Synthetic Polypeptide
DIQLTQSPSFLSASVGDRVTITCRASSGVWYLYWYQQKPGKAPKLWIYFTSTLASGVPSRFSGSGSGNEYTLTISSLQPEDFATYYCQQFTSSPYTFGQGTKLEIK

SEQ ID NO: 129 - protein, 106 aa, Artificial Sequence
  Synthetic Polypeptide
DIQLTQSPSFLSASVGDRVTITCRASSGVYYLYWYQQKPGKAPKLWIYFTSTLASGVPSRFSGSGSGNEYTLTISSLQPEDFATYYCQQFTSSPYTFGQGTKLEIK

SEQ ID NO: 130 - protein, 106 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFTRSPYTFGQGTKLEIK

SEQ ID NO: 131 - protein, 106 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFTSKPYTFGQGTKLEIK

SEQ ID NO: 132 - protein, 106 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFKSSPYTFGQGTKLEIK

SEQ ID NO: 133 - protein, 106 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFRSSPYTFGQGTKLEIK

SEQ ID NO: 134 - protein, 106 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFYSSPYTFGQGTKLEIK

SEQ ID NO: 135 - protein, 213 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFTSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 213 aa, Artificial Sequence
  Synthetic Polypeptide
DIQLTQSPSFLSASVGDRVTITCRASSGVNYLYWYQQKPGKAPKLWIYFTSTLASGVPSRFSGSGSGNEYTLTISSLQPEDFATYYCQQFTSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 213 aa, Artificial Sequence
  Synthetic Polypeptide
DIQLTQSPSFLSASVGDRVTITCRASSGVNYLYWYQQKPGKAPKLWIYFTSTLAPGVPSRFSGSGSGNEYTLTISSLQPEDFATYYCQQFTSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 213 aa, Artificial Sequence
  Synthetic Polypeptide
DIQLTQSPSFLSASVGDRVTITCRASSGVNYLYWYQQKPGKAPKLWIYFTSTLAPGVPSRFSGSGSGNEYTLTISSLQPEDFATYYCQQFTSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 452 aa, Artificial Sequence
  Synthetic Polypeptide
EVQLLESGGGLVQPGGSLRLSCAASGFTFTSYAMSWVRQAPGKGLEWVAVISHGGTYTYYADSVKGRFTISRDNAKNTLYLQMNSLRAEDTAVYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 452 aa, Artificial Sequence
  Synthetic Polypeptide
EVQLLESGGGLVQPGGSLRLSCAASGFTFTSYAMSWVRQAPGKGLEWVATISHGGTYTYYPDSVKGRFTISRDNAKNTLYLQMNSLRAEDTAMYYCARHPIYSGNYQGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2018/0244787 A1
(43) Pub. Date: Aug. 30, 2018

(54) ANTI-HUMAN CXCR3 ANTIBODIES FOR TREATMENT OF VITILIGO

(71) Applicants: SANOFI, Paris (FR); UNIVERSITY OF MASSACHUSETTS, Boston, MA (US)

(72) Inventors: William H. Brondyk, Mansfield, MA (US); Ruiyin Chu, Belle Mead, NJ (US); Timothy D. Connors, Shrewsbury, MA (US); Sunghae Park, Waban, MA (US); Huawei Qiu, Westborough, MA (US); Michele Youd, Lexington, MA (US); John Harris, Boston, MA (US); Jillian Richmond, Boston, MA (US)

(21) Appl. No.: 15/851,651

(22) Filed: Dec. 21, 2017

Related U.S. Application Data
(60) Provisional application No. 62/437,889, filed on Dec. 22, 2016.

(30) Foreign Application Priority Data
Dec. 14, 2017 (EP) 17306770.3

(51) Int. Cl. C07K 16/28; A61P 17/00

(52) U.S. Cl. CPC C07K 16/2866; A61P 17/00; C07K 2317/24; C07K 2317/56; A61K 2039/505; C07K 2317/14; C07K 2317/92; C07K 2317/565; C07K 2317/732; C07K 2317/52

(57) ABSTRACT

Provided are methods and uses of anti-CXCR3 antibodies to treat CXCR3-associated disorders such as vitiligo. In certain embodiments, the anti-CXCR3 antibodies are humanized anti-human CXCR3 antibodies with enhanced effector function against cells expressing CXCR3 on their surface.

### RELATED APPLICATION

This application claims the benefit of U.S. Provisional Patent Application Ser. No. 62/437,889, filed Dec. 22, 2016, and European Patent Application No. 17306770.3, filed Dec. 14, 2017, the entire disclosures of which are hereby incorporated herein by reference.

### BACKGROUND OF THE INVENTION

The invention relates to antibodies and methods of using antibodies to treat disorders associated with CXCR3 signaling, such as vitiligo.

Vitiligo is a disfiguring autoimmune skin disease in which autoreactive CD8⁺ T cells kill melanocytes in the epidermis, resulting in patchy depigmentation. Though it is one of the most common autoimmune diseases, affecting 1% of the population worldwide, currently there are no FDA-approved treatments for vitiligo.

C-X-C motif chemokine receptor 3 (CXCR3) is a chemokine receptor primarily expressed on antigen experienced (memory), effector and activated T cells and is involved in recruiting these T cell subsets to sites of tissue inflammation in response to its primary ligands: CXCL9 (MIG), CXCL10 (IP-10), and CXCL11 (I-TAC). CXCR3 and CXCL10 are expressed in human T1D patients (Uno et al., Endocr J 57: 991-96 (2010); Roep et al., Clin Exp Immunol 159: 338-43 (2003); Tanaka et al., Diabetes 58: 2285-2291 (2009)). In these patients, CXCL10 is expressed in the remaining insulin-producing beta cells in the islets. CXCR3 is expressed on invading T cells surrounding the islets. Similar expression patterns have been reproduced in non-obese diabetic (NOD) mice, a mouse model of type 1 diabetes (Morimoto et al., J Immunol 173: 7017-24 (2004); Li et al., World J Gastroenterol 11(30): 4750-2 (2005); Sarkar et al., Diabetes 61(2):436-46 (2012)).

CXCR3 is expressed by dermal CD3⁺ lymphocytes and plasmacytoid dendritic cells, and its chemokine ligands, CXCL10 and CXCL9, are up-regulated, in psoriatic lesions (Rottman et al., Lab Invest 81(3): 335-47 (2001); Chen et al., Arch Dermatol Res 302(2): 113-23 (2010)). CXCR3 is also expressed on infiltrating T cells present in certain types of inflamed tissues, while CXCL9, CXCL10 and CXCL11 are often produced by local cells in inflammatory lesions.

Upregulation of CXCR3 has been implicated in a range of autoimmune disorders. Largely absent from naive T cells, CXCR3 expression is upregulated upon activation with antigen. CXCR3 recruits these cells, including T helper 1 (Thl) cells, to sites of tissue inflammation in response to its primary ligands. Beta cells in the islets of Langerhans express CXCL9 and CXCL10 (Frigerio at al., Nat Med 8: 1414-20 (2002)), and T cells that have infiltrated the pancreas express CXCR3 (Christen et al., J Immunol 171: 6838-45 (2003); Van Halteren at al., Diabetologia 48: 75-82 (2005); Uno et al., Endocr J 57: 991-96 (2010); Roep et al., Clin Exp Immunol 159: 338-43 (2003); Tanaka et al., Diabetes 58: 2285-2291 (2009); Sarkar et al., Diabetes 61(2):436-46 (2012)). U.S. Pat. No. 8,865,870 to Youd et al. describes anti-CXCR3 antibodies.

In view of the prevalence of vitiligo and other disorders in which CXCR3 has been implicated, a need exists for additional methods that target CXCR3 signaling, e.g., to treat or reduce the progression of a disorder such as vitiligo in a patient.

### SUMMARY OF THE INVENTION

Provided herein are methods of treatment of CXCR3-associated diseases and disorders, such as vitiligo, by administering humanized antibodies or antigen-binding fragments thereof that specifically bind CXCR3. In certain embodiments, the anti-CXCR3 antibodies provided for the methods and uses herein have the capability of directing depletion of CXCR3-expressing cells, or may be engineered with enhanced capability of directing depletion of CXCR3-expressing cells to treat CXCR3-associated diseases and disorders such as vitiligo.

In some embodiments, the humanized antibodies or antigen-binding fragments thereof provided for the methods and uses herein have a germinality score of at least 0.885 when comparing all residues of the VH and VL chain except D-region residues of VH) or at least 0.950 (when comparing residues of the framework regions only as determined by IMTG). In some embodiments, the humanized antibodies or antigen-binding fragments thereof provided herein have a KD of at least 1×10 M. In some embodiments, the humanized antibodies or antigen-binding fragments thereof provided herein have a kd of less than 7×10⁻⁵ 1/Ms. In some embodiments, the humanized antibodies or antigen-binding fragments thereof provided herein have a germinality score of at least 0.885 when comparing all residues of the VH and VL chain except D-region residues of VH) or at least 0.950 (when comparing residues of the framework regions only as determined by IMTG) and a KD of at least 1×10 M, and a germinality score of at least 0.885 when comparing all residues of the VH and VL chain except D-region residues of VH) or at least 0.950 (when comparing residues of the framework regions only as determined by IMTG). In some embodiments, the humanized antibodies or antigen-binding fragments thereof provided herein have a germinality score of at least 0.885 when comparing all residues of the VH and VL chain except D-region residues of VH) or at least 0.950 (when comparing residues of the framework regions only as determined by IMTG), KD of at least 1×10 M, and a kd of less than 7×10⁻⁵ 1/Ms.

In certain embodiments, humanized CXCR3 antibodies comprising particular light chain variable regions paired with particular heavy chain variable regions are provided. In certain embodiments, the humanized CXCR3 antibodies provided herein comprise a variant human IgG1 Fc region which confers enhanced effector function against cells expressing human CXCR3 on their surface.

In a first aspect, provided herein are humanized anti-human CXCR3 antibodies or pharmaceutical compositions thereof for use in a method of depleting CXCR3 expressing cells in a subject, wherein the humanized anti-human CXCR3 antibodies comprise a heavy chain (HC) having a heavy chain variable region (VH) and a light chain (LC) having a light chain variable region (VL). In some embodiments, CD4+ T-cells are depleted. In some embodiments, CD8+ T-cells are depleted. In some embodiments, CD4+ and CD8+ T cells are depleted. In some embodiments, CD4+ memory T-cells are depleted. In some embodiments, CD8+ memory T-cells are depleted. In some embodiments, CD4+ memory T-cells and CD8+ memory T-cells are depleted. In one embodiment, the subject has a T-cell-mediated autoimmune disease. In another embodiment, the subject has vitiligo.

In one embodiment of the first aspect, the VH and VL of the humanized anti-human CXCR3 antibodies provided herein comprise amino acid sequences of sequence pairs shown in Table 1 and the HC further comprises a human IgG1 Fc region comprising an amino acid sequence of any one of SEQ ID NOs: 2, 3, 4, 5, 6, 7, 8, 9, 10, or 11.

TABLE 1

| VH | VL | VH | VL | VH | VL |
|---|---|---|---|---|---|
| SEQ ID | SEQ ID | SEQ ID | SEQ ID | SEQ ID | SEQ ID |
| NO: | NO: | NO: | NO: | NO: | NO: |
| 20 | 24 | 94 | 126 | 115 | 24 |
| 18 | 22 | 95 | 126 | 116 | 126 |
| 80 | 130 | 96 | 126 | 116 | 130 |
| 81 | 24 | 97 | 126 | 116 | 24 |
| 82 | 130 | 98 | 126 | 117 | 126 |
| 82 | 24 | 99 | 126 | 118 | 126 |
| 83 | 126 | 100 | 126 | 20 | 123 |
| 83 | 130 | 101 | 126 | 20 | 124 |
| 83 | 24 | 102 | 126 | 20 | 125 |
| 84 | 126 | 103 | 126 | 20 | 126 |
| 84 | 24 | 104 | 126 | 20 | 127 |
| 85 | 24 | 105 | 126 | 20 | 128 |
| 86 | 126 | 106 | 127 | 20 | 129 |
| 87 | 126 | 108 | 127 | 20 | 130 |
| 87 | 133 | 109 | 126 | 20 | 131 |
| 87 | 24 | 110 | 127 | 20 | 132 |
| 88 | 24 | 111 | 126 | 20 | 133 |
| 89 | 24 | 112 | 126 | 20 | 134 |
| 90 | 126 | 121 | 24 | 119 | 126 |
| 91 | 126 | 113 | 130 | 122 | 126 |
| 91 | 130 | 113 | 24 | 120 | 130 |
| 92 | 130 | 114 | 130 | | |
| 92 | 24 | 115 | 126 | | |
| 93 | 126 | 115 | 130 | | |

In another embodiment of the first aspect, the VH of the humanized anti-human CXCR3 antibodies provided herein comprises an amino acid sequence of SEQ ID NO:20 and the VL comprises an amino acid sequence of SEQ ID NO:24. In some embodiments, the humanized the human IgG1 Fc region comprises an amino acid sequence of SEQ ID NO:2, or SEQ ID NO:9, or SEQ ID NO:10, or SEQ ID NO:11.

In another embodiment of the first aspect, the VH of the humanized anti-human CXCR3 antibodies provide herein comprises an amino acid sequence of SEQ ID NO:18 and the VL comprises amino acid sequence of SEQ ID NO:22. In some embodiments, the antibodies comprise a human IgG1 Fc region. In some embodiments, the human IgG1 Fc region comprises an amino acid sequence of SEQ ID NO:2, or SEQ ID NO:9, or SEQ ID NO:10, or SEQ ID NO:11.

In other embodiments of the first aspect, HC and LC of the humanized anti-human CXCR3 antibodies provided herein comprise the amino acid sequences of the SEQ ID NO pairs shown in Table 2.

TABLE 2

| HC | LC | HC | LC | HC | LC |
|---|---|---|---|---|---|
| SEQ ID | SEQ ID | SEQ ID | SEQ ID | SEQ ID | SEQ ID |
| NO: | NO: | NO: | NO: | NO: | NO: |
| 25 | 135 | 139 | 135 | 33 | 137 |
| 26 | 135 | 31 | 137 | 140 | 137 |
| 27 | 135 | 32 | 137 | | |

In another embodiment of the first aspect, the HC of the humanized anti-human CXCR3 antibodies provided herein comprise a human IgG1 Fc region having reduced fucose content. In some embodiments, the humanized anti-human CXCR3 antibodies provided are produced in a host cell that is cultured in media containing a glycosylation inhibitor. In one embodiment, the glycosylation inhibitor is kifunensine.

In a second aspect, provided herein are humanized anti-human CXCR3 antibodies or pharmaceutical compositions thereof for use in a method of treating a T-cell-mediated autoimmune disease wherein the humanized anti-human CXCR3 antibodies comprise a heavy chain (HC) having a heavy chain variable region (VH) and a light chain (LC) having a light chain variable region (VL). In one embodiment, the T-cell-mediated disease is vitiligo.

In a third aspect, provided herein are methods of treating a T-cell-mediated autoimmune disease, comprising administering to a subject in need thereof a humanized anti-human CXCR3 antibody comprising a heavy chain (HC) having a heavy chain variable region (VH) and light chain (LC) having a light chain variable region (VL). In some embodiments, the T-cell-mediated autoimmune disease is vitiligo.

In a fourth aspect, provided herein are methods of treating a T-cell-mediated autoimmune disease, comprising providing instruction to administer to a subject in need thereof a humanized anti-human CXCR3 antibody comprising a heavy chain (HC) having a heavy chain variable region (VH) and light chain (LC) having a light chain variable region (VL). In some embodiments, the T-cell-mediated autoimmune disease is vitiligo.

In a fifth aspect, provided herein are kits for treating a T-cell-mediated autoimmune disease, comprising a humanized anti-human CXCR3 antibody comprising a heavy chain (HC) having a heavy chain variable region (VH) and light chain (LC) having a light chain variable region (VL), and instructions to administer the humanized anti-human CXCR3 to a human subject in need thereof.

In some embodiments of the first, second, third, fourth, and fifth aspects provided herein, the VH and VL of the humanized anti-human CXCR3 antibodies comprise amino acid sequences of sequence pairs shown in Table 1 and the HC comprises a human IgG1 Fc region comprising an amino acid sequence of SEQ ID NOs:2, 3, 4, 5, 6, 7, 8, 9, 10, or 11. In another embodiment, the VH comprises an amino acid sequence of SEQ ID NO:20 and the VL comprises an amino acid sequence of SEQ ID NO:24. In another embodiment, the VH comprises an amino acid sequence of SEQ ID NO:20 and the VL comprises an amino acid sequence of SEQ ID NO:24 and the human IgG1 Fc region comprises an amino acid sequence of SEQ ID NO:2, or SEQ ID NO:9, or SEQ ID NO:10, or SEQ ID NO:11.

In other embodiments of the second, third, fourth, fifth, sixth, and seventh aspects provided herein, the VH comprises an amino acid sequence of SEQ ID NO:18 and the VL comprises an amino acid sequence of SEQ ID NO:22. In some embodiments, the humanized the human IgG1 Fc region comprises an amino acid sequence of SEQ ID NO:2, or SEQ ID NO:9, or SEQ ID NO:10, or SEQ ID NO:11.

In other embodiments of the first, second, third, fourth, and fifth aspects provided herein, the HC and LC of the antibodies comprise the amino acid sequences of the sequence pairs shown in Table 2.

In other embodiments of the first, second, third, fourth, and fifth aspects provided herein, the HC of the humanized anti-human CXCR3 antibodies provided herein comprise a human IgG1 Fc region having reduced fucose content. In some embodiments, the humanized anti-human CXCR3 antibodies provided are produced in a host cell that is cultured in media containing a glycosylation inhibitor. In some embodiments, the glycosylation inhibitor is kifunensine.

### BRIEF DESCRIPTION OF THE DRAWINGS

FIG. 1 is a series of six graphs showing the amount of various T cell subsets in blood following antibody treatment as follows: “Hamster CXCR3-173” (hamster anti-mouse CXCR3), “CXCR3 mIgG2a Dab” (Hamster CXCR3-173 engineered to substitute mouse IgG2a constant region with mutation to remove effector function), “CXCR3 mIgG2a WT” (Hamster CXCR3-173 engineered to substitute mouse wild-type IgG2a constant region), “CXCR3 mIgG1-agly” (Hamster CXCR3-173 engineered to substitute mouse IgG1 constant region with N297G mutation), and “Untreated” represents untreated control.

FIG. 2A is a series of six graphs depicting binding of various Fc-engineered versions of CXCR3-173 by recombinant mouse FcγRI (mFcRI), as measured by surface plasmon resonance (Biacore) binding assay. BMP5, anti-BMP5 mIgG1 isotype control; mIgG1 agly, CXCR3-173 engineered to substitute mouse IgG1 constant region with N297G mutation; mIgG2a WT, CXCR3-173 engineered to substitute mouse wild-type IgG2a constant region; mIgG2a Dab, CXCR3-173 engineered to substitute mouse IgG2a constant region with mutation to remove effector function; mIgG3, CXCR3-173 engineered to substitute mouse wild-type IgG3 constant region; Hamster CXCR3, parent hamster mAb CXCR3-173.

FIG. 2B is a series of six graphs depicting binding of various Fc-engineered versions of CXCR3-173 by recombinant mouse FcγRIIb (mFcRIIb), as measured by Biacore binding assay. The antibody designations are the same as for FIG. 2A.

FIG. 2C is a series of six graphs depicting binding of various Fc-engineered versions of CXCR3-173 by recombinant mouse FcγRIII (mFcRIII), as measured by Biacore binding assay. The antibody designations are the same as for FIG. 2A.

FIG. 2D is a series of six graphs depicting binding of various Fc-engineered versions of CXCR3-173 by recombinant mouse FcγRIV (mFcRIV), as measured by Biacore binding assay. Various antibody designations are the same as for FIG. 2A.

FIG. 3A is a table summarizing structure-effector function characteristics for an anti-human CXCR3 antibody with engineered human IgG1 constant regions.

FIG. 3B is a bar graph depicting in vitro antibody-dependent cellular cytotoxicity (ADCC)-mediated lysis of CHO-human CXCR3 target cells with various anti-human CXCR3 antibodies at the indicated concentrations and 5:1 effector to target (E:T) ratio. Effector cells are natural killer (NK) cells from a single donor. IgG, human IgG1 isotype control. Anti-human CXCR3 mAb tested were clone 4 (CXCR3 CL4), clone 12 (CXCR3 CL12), clone 82 (CXCR3 CL82), clone 135 (CXCR3 CL135), 53Hu37, and engineered Fc variants of 53Hu37 M1, M2, and M3 as described in FIG. 3A. Kif, kifunensine treatment. ALEM, alemtuzumab.

FIG. 3C is a bar graph depicting in vitro ADCC-mediated lysis of CHO-human CXCR3 target cells with various antibodies at the indicated concentrations and 3:1 Effector to Target cell (E:T) ratio. Effector cells are from an NK-like cell line (NK92-CD16V). IgG, human IgG1 isotype control. Anti-human CXCR3 mAb tested were 53Hu37 and the engineered Fc variant M1 as in FIG. 3A. CXCR3 CL4, anti-human CXCR3 mAb clone 4. Kif, kifunensine-treatment. ALEM is alemtuzumab.

FIG. 4 is a table summarizing Biacore data for 53Hu37 and the indicated variants showing binding affinity, KD, to human FcγRIIa (rhFcγRIIa), human FcγRIII-F158 (rhFcγRIII-F158), human FcγRIII-V158 (rhFcγRIII-V158), and mouse FcγRIV (rmFcγRIV). M1-M3 are as described in FIG. 3A and kif defucosylated 53Hu37.

FIG. 5A is a series of six graphs depicting depletion of indicated T-cell subsets in vivo in cynomolgus monkeys treated with indicated antibodies administered at a dose of 2 mg/kg body weight. M1: S239D/I332E variant of 53Hu37; Kif: defucosylated 53Hu37; Veh: vehicle control. N=8 for CXCR3 antibody groups. N=6 for vehicle groups.

FIG. 5B is a graph depicting combined pharmacokinetic data assessing concentration of indicated antibodies in sera of cynomolgus monkeys treated the indicated amount of time beforehand with a single dose of indicated antibodies administered at a dose of 2 mg/kg body weight. Anti-human CXCR3 mAb tested were 53Hu37, kifunensine-treated 53Hu37 (53Hu37 kif), and M1 variant of 53Hu37 (53Hu37 M1).

FIG. 6 is a table showing the SEQ ID Nos and corresponding sequences.

### DETAILED DESCRIPTION

Reference will now be made in detail to certain exemplary embodiments according to the present disclosure, certain examples of which are illustrated in the accompanying drawings.

Provided herein are methods and uses for humanized antibodies or antigen-binding fragments thereof that specifically bind human CXCR3. In certain embodiments, the anti-CXCR3 antibodies provided herein have the capability of directing depletion of CXCR3-expressing cells, or are engineered with enhanced capability of directing depletion of CXCR3-expressing cells to treat CXCR3-associated diseases and disorders. In some embodiments, therapies are disclosed for targeting CXCR3 to treat vitiligo

### Antibodies

As used herein, the term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated V_{H} or VH) and a heavy chain constant region (C_{H} or CH). The heavy chain constant region comprises three domains, C_{H}1, C_{H}2 and C_{H}3. The Fc portion of the heavy chain comprises C_{H}2 and C_{H}3.

Each light chain comprises a light chain variable region (abbreviated V_{L}) and a light chain constant region (C_{L} or CL). The light chain constant region comprises one domain (CL¹).

The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, and FR4.

As used herein, the term “antigen-binding fragment” of an antibody includes any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units comprising the amino acid residues that mimic the hypervariable region of an antibody (e.g., an isolated complementarity determining region (CDR)). Other engineered molecules, such as bi-, tri-, tetra-specific antibodies, and minibodies, are also encompassed within the expression “antigen-binding fragment.”

In certain embodiments, a CXCR3 antibody or antigen-binding fragment comprises at least one antigen-binding domain. In some embodiments, the antibody or fragment is multi-specific and comprises two or more (e.g., 2, 3, 4, 5, or more) antigen-binding domains, such that the antibody or fragment is capable of binding two or more CXCR3 molecules at the same or different epitopes, or capable of binding to CXCR3 and at least one other antigen with high affinity. The antigen-binding portion can comprise one or more fragments of an antibody that retains the ability to specifically bind to an antigen. These fragments may comprise the heavy and/or light chain variable region from a parent antibody or from a variant of a parent antibody.

As used herein, the term “antigen” refers to the binding site or epitope recognized by an antibody or antigen-binding fragment thereof.

The “epitope” or “antigenic determinant” is a portion of an antigen molecule that is responsible for specific interactions with the antigen-binding domain of an antibody.

As used herein, “binds” with respect to an antibody or antigen-binding fragment thereof refers to the ability of the antibody or antigen-binding fragment to form one or more noncovalent bonds with a cognate antigen, by noncovalent interactions between the antibody combining sites of the antibody and the antigen. The antigen can be an isolated antigen or can be presented in association with another entity, such as in the context of a polypeptide on the surface of a cell.

As used herein, the term “specifically binds to” refers to the ability of an antibody or an antigen-binding fragment thereof to bind to an antigen with an Kd of at least about 1×10⁻⁶ M, 1×10⁻⁷ M, 1×10⁻⁸M, 1×10⁻⁹ M, 1×10⁻¹⁰ M, 1×10⁻¹¹ M, 1×10⁻¹² M, or more. In certain embodiments, the term refers to the ability of an antibody or an antigen-binding fragment thereof to bind to an antigen with an affinity that is at least two-fold greater than its affinity for a nonspecific antigen. It shall be understood, however, that an antibody or an antigen-binding fragment thereof, is capable of specifically binding to two or more antigens which are related in sequence (e.g., human and cynomolgous CXCR3). Non-specific binding usually has a low affinity with a moderate to high capacity. If necessary, non-specific binding can be reduced without substantially affecting specific binding by varying the binding conditions. Such conditions are known in the art, and a skilled artisan using routine techniques can select appropriate conditions. The conditions are usually defined in terms of concentration of antibodies, ionic strength of the solution, temperature, time allowed for binding, and concentration of blocking molecules such as serum albumin and milk casein.

Affinity constants can be determined by standard kinetic methodology for antibody reactions, for example, immunoassays (e.g., ELISA), or surface plasmon resonance (SPR). Instrumentation and methods for real time detection and monitoring of binding rates are known and are commercially available (e.g., Biacore 2000, Biacore AB, Upsala, Sweden and GE Healthcare Life Sciences).

As used herein, a “complementarity-determining region” or “CDR” refers to one of a plurality of portions within each variable region of an antibody or antigen-binding fragment that together form an antigen-binding site of an antibody. Each variable region domain contains three CDRs, named CDR1, CDR2 and CDR3. Accordingly, the variable heavy chain domain (VH) comprises CDR-H1, CDR-H2 and CDR-H3, and the variable light chain domain (VL) comprises CDR-L1, CDR-L2, and CDR-L3. The three CDRs are non-contiguous along the linear amino acid sequence, but are proximate in the folded polypeptide. The CDRs are located within the loops that join the parallel strands of the beta sheets of the variable domain.

As used herein the term “framework (FR) amino acid residues” refers to those amino acids in the framework region of an Ig chain. The term “framework region” or “FR region” as used herein, includes the amino acid residues that are part of the variable region, but are not part of the CDRs. Therefore, a variable region framework is between about 100-120 amino acids in length but includes only those amino acids outside of the CDRs.

As used herein, the term “% identical” or “percent identical” means that in a comparison of two sequences over a specified region, the two sequences have the specified number of identical residues in the same position. The term “% similar” or “percent similar” has a similar meaning but in addition to the number of identical amino acids between the two sequences, regard is also given to where the amino acids are not identical but are conservative substitutions. Percentage identity can be determined using known computer algorithms such as BLASTP, BLASTN, and the FASTA program (Altschul, S F, et al., J Mol Biol 215: 403 (1990)), using, for example, the default parameters as in Pearson et al., Proc Natl Acad Sci USA 85: 2444 (1988). For example, the BLAST function of the National Center for Biotechnology Information (NCBI) database can be used to determine identity.

In certain embodiments, the antibodies provided herein are humanized antibodies. “Humanized antibodies” are antibody molecules that bind the desired antigen, have one or more CDRs from a non-human species (e.g., a mouse antibody), and have at least some portion of the framework regions and/or constant domains from a human immunoglobulin molecule. Known human Ig sequences are disclosed in, e.g., ncbi.nlm.nih.gov/entrez-/query.fcgi; atcc.org/phage/hdb.html; sciquest.com; abcam.com; antibodyresource.com/onlinecomp.html; and Kabat et al., Sequences of Proteins of Immunological Interest, U.S. Dept. Health (1983). Imported human sequences can be used to reduce immunogenicity or reduce, enhance or modify binding, affinity, on-rate, off-rate, avidity, specificity, half-life, or any other suitable characteristic, as known in the art. Art recognized methods for antibody humanization are described in Jones et al., Nature 321: 522 (1986); Verhoeyen et al., Science 239: 1534 (1988); Sims et al., J Immunol 151: 2296 (1993); Chothia and Lesk, J Mol Biol 196: 901 (1987); Carter et al., Proc Natl Acad Sci USA 89: 4285 (1992); Presta et al., J Immunol 151: 2623 (1993); U.S. Pat. Nos. 5,589,205; 5,565,332; 6,180,370; 6,632,927; 7,241,877; 7,244,615; 7,244,832; 7,262,050; and U.S. Patent Publication No. 2004/0236078 (filed Apr. 30, 2004), which are incorporated herein by reference in their entirety.

In certain embodiments, certain framework residues in the humanized antibodies provided herein have been substituted with the corresponding residue from the CDR donor antibody, e.g., substituted with framework residues from a mouse anti-human CXCR3 antibody, in order to alter, e.g., improve, antigen-binding. These framework substitutions have been identified by modeling of the interactions of the CDR and framework residues to identify framework residues important for antigen-binding and sequence comparison to identify unusual framework residues at particular positions. In some embodiments, 4D humanization was used to prepare humanized antibody variants of the present disclosure. See WO 2009/032661 (which is incorporated herein by reference in its entirety), e.g., at paragraphs [0037]-[0044] for methods used in 4D humanization. Briefly, 4D humanization can comprise: (a) building a 3-D model of the variable domain that is to be humanized; (b) identifying the flexible residues in the variable domain using a molecular dynamics simulation of the 3-D model of the domain; (c) identifying the closest human germline by comparing the molecular dynamics trajectory of the 3-D model to the molecular dynamics trajectories of 49 human germlines; and (d) mutating the flexible residues, which are not part of the CDR, into their human germline counterpart (as identified in step (c)).

In some embodiments, humanized CXCR3 antibodies, or antigen-binding fragments thereof, comprising the VH and VL sequences set forth in Table 1 are provided.

In some embodiments, humanized CXCR3 antibodies, or antigen-binding fragments thereof, comprising the heavy chain (HC) and light chain (LC) sequences set forth in Table 2 are provided.

### Antibody Effector Function/Depleting Activity

In certain embodiments, the anti-CXCR3 antibodies disclosed herein have the capability of directing depletion of CXCR3-expressing cells, or may be engineered with enhanced capability of directing depletion of CXCR3-expressing cells to treat CXCR3-associated diseases and disorders. CXCR3-expressing cells that can be depleted by the antibodies disclosed herein can include CD4⁺ T cells and/or CD8⁺ T cells. The CXCR3-expressing cells that can be depleted by the antibodies disclosed herein can include CD4⁺ memory T cells and/or CD8⁺ memory T cells. As used herein, “depletion” with respect to CXCR3⁺ cells (i.e., cells expressing CXCR3 on their cell surface) refers to the removal of these cells from a population of cells. Reference to depletion includes complete or partial depletion. Further, depletion may be permanent or temporary, and may be to varying extents in magnitude and/or location. Depletion may be the result of cell death, such as by apoptosis or necrosis. Depletion can be assessed by measuring the number of CXCR3⁺ cells in a population using any method known in the art (e.g., flow cytometry, immunohistochemistry, etc.), before and after exposure to an antibody or antigen-binding fragment provided herein, or in the absence and presence of an antibody or antigen-binding fragment provided herein. Following exposure to an antibody or antigen-binding fragment provided herein, CXCR3⁺ cells can be depleted by at least or about 10%, 15%, 20%, 25%, 30%, 35%, 40%, 45%, 50%, 55%, 60%, 65%, 70%, 75%, 80%, 85%, 90%, 95%, or more.

In certain embodiments, the humanized anti-human CXCR3 antibody exhibits enhanced effector function against cells expressing human CXCR3 on their surface compared to a corresponding humanized anti-human CXCR3 antibody with wild-type Fc region, e.g., wild-type human IgG1 Fc. As used herein, “enhanced effector function” refers to measurably increased ability of an antibody to direct any one or more of antibody-dependent cellular cytotoxicity (ADCC), complement-mediated cytotoxicity (CDC), or antibody-dependent cell-mediated phagocytosis (ADCP) against a suitable target cell, as compared to a reference antibody, under the same conditions, having the same antigen specificity and wild-type human IgG1 Fc region. In certain embodiments, the reference antibody comprises a variant human Fc region. In certain embodiments, the effector function is ADCC, ADCP, or CDC, or any combination thereof. In certain embodiments, the effector function is ADCC, or CDC, or both ADCC and CDC. In certain embodiments, the effector function is ADCC. In certain embodiments, the effector function is CDC. In certain embodiments, the effector function is both ADCC and CDC. In certain embodiments, the effector function is ADCP.

As used herein, a “variant human IgG1 Fc region” refers to a human IgG1 Fc region that has been engineered or modified to include one or more amino acid mutations or amino acid modifications compared to wild-type human IgG1 Fc. In certain embodiments, the wild-type human IgG1 Fc region comprises the amino acid sequence SLGTQTYICNVNHKPSNTKVDKKVEPKSCDKTHTCPPCPAPELLGGPSVFLFPPKPKDTL MISRTPEVTCVVVDVSHEDPEVKFNWYVDGVEVHNAKTKPREEQYNSTYRVVSVLTVL HQDWLNGKEYKCKVSNKALPAPIEKTISKAKGQPREPQVYTLPPSRDELTKNQVSLTCL VKGFYPSDIAVEWESNGQPENNYKTTPPVLDSDGSFFLYSKLTVDKSRWQQGNVFSCSV MHEALHNHYTQKSLSLSPG (Eu numbering 192-446) (SEQ ID NO:2).

In certain embodiments, the variant human IgG1 Fc region comprises at least one of the following amino acid substitutions: G236A, S239D, S267E, H268F, S324T, I332E (Eu numbering), or any combination thereof. In certain embodiments, the variant human IgG1 Fc region comprises at least one of the following sets of amino acid substitutions: S239D/I332E, G236A/S267E/H268F/S324T/I332E, and S239D/H268F/S324T/I332E (Eu numbering). In certain embodiments, the variant human IgG1 Fc region comprises the amino acid substitutions S239D/I332E. In other embodiments, the variant human IgG1 Fc region comprises the amino acid substitutions G236A/S267E/H268F/S324T/I332E. In still other embodiments, the variant human IgG1 Fc region comprises the amino acid substitutions S239D/H268F/S324T/I332E.

For example, in certain embodiments, the variant human IgG1 Fc region comprises an amino acid sequence of SEQ ID NOs:3, 4, 5, 6, 7 or 8.

In certain embodiments, the variant human IgG1 Fc region comprises a sequence at least 90 percent identical to any one or more of SEQ ID NOs: 3-8, provided that, in each instance, the specified amino acid substitution is maintained. In various embodiments, the variant human IgG1 Fc region comprises a sequence at least 90 percent identical, at least 91 percent identical, at least 92 percent identical, at least 93 percent identical, at least 94 percent identical, at least 95 percent identical, at least 96 percent identical, at least 97 percent identical, at least 98 percent identical, or at least 99 percent identical to any one or more of SEQ ID NOs:3-8, provided that, in each instance, the specified amino acid substitution is maintained.

In certain embodiments, the variant human IgG1 Fc region comprises at least one of the following sets of amino acid substitutions: S239D/I332E, G236A/S267E/H268F/S324T/I332E, and S239D/H268F/S324T/I332E (Eu numbering). For example, in certain embodiments, the variant human IgG1 Fc region comprises an amino acid sequence of SEQ ID NOs: 9, 10, or 11.

In certain embodiments, at least one amino acid substitution is S239D/I332E (Eu numbering). In other embodiments, at least one amino acid substitution is G236A/S267E/H268F/S324T/I332E (Eu numbering). In certain embodiments, the at least one amino acid substitution is S239D/H268F/S324T/I332E (Eu numbering).

In certain embodiments, the variant human IgG1 Fc region of the humanized anti-CXCR3 antibodies provided herein comprises a sequence at least 90 percent identical to any one of SEQ ID NOs: 9-11, provided that, in each instance, the specified amino acid substitutions and enhanced effector function are maintained. are maintained. In various embodiments, the variant human IgG1 Fc region comprises a sequence at least 90 percent identical, at least 91 percent identical, at least 92 percent identical, at least 93 percent identical, at least 94 percent identical, at least 95 percent identical, at least 96 percent identical, at least 97 percent identical, at least 98 percent identical, or at least 99 percent identical to any one or more of SEQ ID NOs:9-11, provided that, in each instance, the specified amino acid substitutions are maintained.

### CDR Variants:

In addition to the foregoing embodiments, provided herein are anti-CXCR3 antibodies comprising 6 CDRs wherein the VH comprises CDRs having amino acid sequence of:

(i) SEQ ID NO:12, SEQ ID NO:35, and SEQ ID NO:14;

(ii) SEQ ID NO:12, SEQ ID NO:35, and SEQ ID NO:45;

(iii) SEQ ID NO:12, SEQ ID NO:36, and SEQ ID NO:45;

(iv) SEQ ID NO:12, SEQ ID NO:37, and SEQ ID NO:14;

(v) SEQ ID NO:12, SEQ ID NO:37, and SEQ ID NO:45;

(vi) SEQ ID NO:12, SEQ ID NO:37, and SEQ ID NO:46

(vii) SEQ ID NO:12, SEQ ID NO:38, and SEQ ID NO:14;

(viii) SEQ ID NO:12, SEQ ID NO:38, and SEQ ID NO:45;

(ix) SEQ ID NO:12, SEQ ID NO:39, and SEQ ID NO:14;

(x) SEQ ID NO:12, SEQ ID NO:39, and SEQ ID NO:47;

(xi) SEQ ID NO:12, SEQ ID NO:40, and SEQ ID NO:14;

(xii) SEQ ID NO:12, SEQ ID NO:13, and SEQ ID NO:45

(xiii) SEQ ID NO:12, SEQ ID NO:13, and SEQ ID NO:46;

(xiv) SEQ ID NO:12, SEQ ID NO:13, and SEQ ID NO:48;

(xv) SEQ ID NO:12, SEQ ID NO:13, and SEQ ID NO:49;

(xvi) SEQ ID NO:12, SEQ ID NO:13, and SEQ ID NO:50;

(xvii) SEQ ID NO:12, SEQ ID NO:13, and SEQ ID NO:51;

(xviii) SEQ ID NO:12, SEQ ID NO:13, and SEQ ID NO:52;

(xix) SEQ ID NO:12, SEQ ID NO:13, and SEQ ID NO:53;

(xx) SEQ ID NO:12, SEQ ID NO:13, and SEQ ID NO:54;

(xxi) SEQ ID NO:12, SEQ ID NO:13, and SEQ ID NO:55;

(xxii) SEQ ID NO:12, SEQ ID NO:13, and SEQ ID NO:56;

(xxiii) SEQ ID NO:12, SEQ ID NO:13, and SEQ ID NO:57;

(xxiv) SEQ ID NO:12, SEQ ID NO:13, and SEQ ID NO:58;

(xxv) SEQ ID NO:12, SEQ ID NO:13, and SEQ ID NO:59;

(xxvi) SEQ ID NO:12, SEQ ID NO:13, and SEQ ID NO:60;

(xxvii) SEQ ID NO:12, SEQ ID NO:13, and SEQ ID NO:61;

(xxviii) SEQ ID NO:12, SEQ ID NO:13, and SEQ ID NO:62;

(xxix) SEQ ID NO:12, SEQ ID NO:13, and SEQ ID NO:63;

(xxx) SEQ ID NO:12, SEQ ID NO:13, and SEQ ID NO:64;

(xxxi) SEQ ID NO:12, SEQ ID NO:13, and SEQ ID NO:65;

(xxxii) SEQ ID NO:12, SEQ ID NO:13, and SEQ ID NO:66;

(xxxiii) SEQ ID NO:34, SEQ ID NO:13, and SEQ ID NO:14;

(xxxiv) SEQ ID NO:12, SEQ ID NO:41, and SEQ ID NO:14;

(xxxv) SEQ ID NO:12, SEQ ID NO:41, and SEQ ID NO:46;

(xxxvi) SEQ ID NO:12, SEQ ID NO:42, and SEQ ID NO:14;

(xxxvii) SEQ ID NO:12, SEQ ID NO:42, and SEQ ID NO:45;

(xxxviii) SEQ ID NO:12, SEQ ID NO:43, and SEQ ID NO:14;

(xxxix) SEQ ID NO:12, SEQ ID NO:44, and SEQ ID NO:14;

(xl) SEQ ID NO:12, SEQ ID NO:41, and SEQ ID NO:14; or

(xli) SEQ ID NO:12, SEQ ID NO:13, and SEQ ID NO:68; and

wherein the VL comprises CDRs having amino acid sequence of:

(i) SEQ ID NO:69, SEQ ID NO:16, and SEQ ID NO:17;

(ii) SEQ ID NO:70, SEQ ID NO:16, and SEQ ID NO:17;

(iii) SEQ ID NO:71, SEQ ID NO:16, and SEQ ID NO:17;

(iv) SEQ ID NO:71, SEQ ID NO:16, and SEQ ID NO:75;

(v) SEQ ID NO:72, SEQ ID NO:16, and SEQ ID NO:17;

(vi) SEQ ID NO:73, SEQ ID NO:16, and SEQ ID NO:17;

(vii) SEQ ID NO:74, SEQ ID NO:16, and SEQ ID NO:17;

(viii) SEQ ID NO:15, SEQ ID NO:16, and SEQ ID NO:75;

(ix) SEQ ID NO:15, SEQ ID NO:16, and SEQ ID NO:76;

(x) SEQ ID NO:15, SEQ ID NO:16, and SEQ ID NO:77;

(xi) SEQ ID NO:15, SEQ ID NO:16, and SEQ ID NO:78; or

(xii) SEQ ID NO:15, SEQ ID NO:16, and SEQ ID NO:79.

In addition to the foregoing embodiments, provided herein are anti-CXCR3 antibodies comprising 6 CDRs wherein the VH comprises CDRs having amino acid sequence of:

(i) SEQ ID NO:12, SEQ ID NO:35, and SEQ ID NO:14;

(ii) SEQ ID NO:12, SEQ ID NO:35, and SEQ ID NO:45;

(iii) SEQ ID NO:12, SEQ ID NO:36, and SEQ ID NO:45;

(iv) SEQ ID NO:12, SEQ ID NO:37, and SEQ ID NO:14;

(v) SEQ ID NO:12, SEQ ID NO:37, and SEQ ID NO:45;

(vi) SEQ ID NO:12, SEQ ID NO:37, and SEQ ID NO:46;

(vii) SEQ ID NO:12, SEQ ID NO:38, and SEQ ID NO:14;

(viii) SEQ ID NO:12, SEQ ID NO:38, and SEQ ID NO:45;

(ix) SEQ ID NO:12, SEQ ID NO:39, and SEQ ID NO:14;

(x) SEQ ID NO:12, SEQ ID NO:39, and SEQ ID NO:47; or

(xi) SEQ ID NO:12, SEQ ID NO:40, and SEQ ID NO:14; and

wherein the VL comprises CDRs having amino acid sequence of:

(i) SEQ ID NO:69, SEQ ID NO:16, and SEQ ID NO:17;

(ii) SEQ ID NO:70, SEQ ID NO:16, and SEQ ID NO:17;

(iii) SEQ ID NO:71, SEQ ID NO:16, and SEQ ID NO:17;

(iv) SEQ ID NO:71, SEQ ID NO:16, and SEQ ID NO:100;

(v) SEQ ID NO:72, SEQ ID NO:16, and SEQ ID NO:17;

(vi) SEQ ID NO:73, SEQ ID NO:16, and SEQ ID NO:17;

(vii) SEQ ID NO:74, SEQ ID NO:16, and SEQ ID NO:17;

(viii) SEQ ID NO:15, SEQ ID NO:16, and SEQ ID NO:75;

(ix) SEQ ID NO:15, SEQ ID NO:16, and SEQ ID NO:76;

(x) SEQ ID NO:15, SEQ ID NO:16, and SEQ ID NO:77;

(xi) SEQ ID NO:15, SEQ ID NO:16, and SEQ ID NO:78; or

(xii) SEQ ID NO:15, SEQ ID NO:16, and SEQ ID NO:79.

In addition to the foregoing embodiments, provided herein are anti-CXCR3 antibodies comprising 6 CDRs wherein the VH comprises CDRs having amino acid sequence of:

(i) SEQ ID NO:12, SEQ ID NO:13, and SEQ ID NO:45

(ii) SEQ ID NO:12, SEQ ID NO:13, and SEQ ID NO:46;

(iii) SEQ ID NO:12, SEQ ID NO:13, and SEQ ID NO:48;

(iv) SEQ ID NO:12, SEQ ID NO:13, and SEQ ID NO:49;

(v) SEQ ID NO:12, SEQ ID NO:13, and SEQ ID NO:50;

(vi) SEQ ID NO:12, SEQ ID NO:13, and SEQ ID NO:51;

(vii) SEQ ID NO:12, SEQ ID NO:13, and SEQ ID NO:52;

(viii) SEQ ID NO:12, SEQ ID NO:13, and SEQ ID NO:53;

(ix) SEQ ID NO:12, SEQ ID NO:13, and SEQ ID NO:54;

(x) SEQ ID NO:12, SEQ ID NO:13, and SEQ ID NO:55; or

(xi) SEQ ID NO:12, SEQ ID NO:13, and SEQ ID NO:56; and

wherein the VL comprises CDRs having amino acid sequence of:

(i) SEQ ID NO:69, SEQ ID NO:16, and SEQ ID NO:17;

(ii) SEQ ID NO:70, SEQ ID NO:16, and SEQ ID NO:17;

(iii) SEQ ID NO:71, SEQ ID NO:16, and SEQ ID NO:17;

(iv) SEQ ID NO:71, SEQ ID NO:16, and SEQ ID NO:75;

(v) SEQ ID NO:72, SEQ ID NO:16, and SEQ ID NO:17;

(vi) SEQ ID NO:73, SEQ ID NO:16, and SEQ ID NO:17;

(vii) SEQ ID NO:74, SEQ ID NO:16, and SEQ ID NO:17;

(viii) SEQ ID NO:15, SEQ ID NO:16, and SEQ ID NO:75;

(ix) SEQ ID NO:15, SEQ ID NO:16, and SEQ ID NO:76;

(x) SEQ ID NO:15, SEQ ID NO:16, and SEQ ID NO:77;

(xi) SEQ ID NO:15, SEQ ID NO:16, and SEQ ID NO:78; or

(xii) SEQ ID NO:15, SEQ ID NO:16, and SEQ ID NO:79.

In addition to the foregoing embodiments, provided herein are anti-CXCR3 antibodies comprising 6 CDRs wherein the VH comprises CDRs having amino acid sequence of:

(i) SEQ ID NO:12, SEQ ID NO:13, and SEQ ID NO:57;

(ii) SEQ ID NO:12, SEQ ID NO:13, and SEQ ID NO:58;

(iii) SEQ ID NO:12, SEQ ID NO:13, and SEQ ID NO:59;

(iv) SEQ ID NO:12, SEQ ID NO:13, and SEQ ID NO:60;

(v) SEQ ID NO:12, SEQ ID NO:13, and SEQ ID NO:61;

(vi) SEQ ID NO:12, SEQ ID NO:13, and SEQ ID NO:62;

(vii) SEQ ID NO:12, SEQ ID NO:13, and SEQ ID NO:63;

(viii) SEQ ID NO:12, SEQ ID NO:13, and SEQ ID NO:64;

(ix) SEQ ID NO:12, SEQ ID NO:13, and SEQ ID NO:65; or

(x) SEQ ID NO:12, SEQ ID NO:13, and SEQ ID NO:66; and

wherein the VL comprises CDRs having amino acid sequence of:

(i) SEQ ID NO:69, SEQ ID NO:16, and SEQ ID NO:17;

(ii) SEQ ID NO:70, SEQ ID NO:16, and SEQ ID NO:17;

(iii) SEQ ID NO:71, SEQ ID NO:16, and SEQ ID NO:17;

(iv) SEQ ID NO:71, SEQ ID NO:16, and SEQ ID NO:75;

(v) SEQ ID NO:72, SEQ ID NO:16, and SEQ ID NO:17;

(vi) SEQ ID NO:73, SEQ ID NO:16, and SEQ ID NO:17;

(vii) SEQ ID NO:74, SEQ ID NO:16, and SEQ ID NO:17;

(viii) SEQ ID NO:15, SEQ ID NO:16, and SEQ ID NO:75;

(ix) SEQ ID NO:15, SEQ ID NO:16, and SEQ ID NO:76;

(x) SEQ ID NO:15, SEQ ID NO:16, and SEQ ID NO:77;

(xi) SEQ ID NO:15, SEQ ID NO:16, and SEQ ID NO:78; or

(xii) SEQ ID NO:15, SEQ ID NO:16, and SEQ ID NO:79.

In addition to the foregoing embodiments, provided herein are anti-CXCR3 antibodies comprising 6 CDRs wherein the VH comprises CDRs having amino acid sequence of:

(i) SEQ ID NO:34, SEQ ID NO:13, and SEQ ID NO:14;

(ii) SEQ ID NO:12, SEQ ID NO:41, and SEQ ID NO:14;

(iii) SEQ ID NO:12, SEQ ID NO:41, and SEQ ID NO:71;

(iv) SEQ ID NO:12, SEQ ID NO:42, and SEQ ID NO:14;

(v) SEQ ID NO:12, SEQ ID NO:42, and SEQ ID NO:70;

(vi) SEQ ID NO:12, SEQ ID NO:43, and SEQ ID NO:14;

(vii) SEQ ID NO:12, SEQ ID NO:44, and SEQ ID NO:14;

(viii) SEQ ID NO:12, SEQ ID NO:41, and SEQ ID NO:14; or

(ix) SEQ ID NO:12, SEQ ID NO:13, and SEQ ID NO:68; and

wherein the VL comprises CDRs having amino acid sequence of:

(i) SEQ ID NO:69, SEQ ID NO:16, and SEQ ID NO:17

(ii) SEQ ID NO:70, SEQ ID NO:16, and SEQ ID NO:17;

(iii) SEQ ID NO:71, SEQ ID NO:16, and SEQ ID NO:17;

(iv) SEQ ID NO:71, SEQ ID NO:16, and SEQ ID NO:75;

(v) SEQ ID NO:72, SEQ ID NO:16, and SEQ ID NO:17;

(vi) SEQ ID NO:73, SEQ ID NO:16, and SEQ ID NO:17;

(vii) SEQ ID NO:74, SEQ ID NO:16, and SEQ ID NO:17;

(viii) SEQ ID NO:15, SEQ ID NO:16, and SEQ ID NO:75;

(ix) SEQ ID NO:15, SEQ ID NO:16, and SEQ ID NO:76;

(x) SEQ ID NO:15, SEQ ID NO:16, and SEQ ID NO:77;

(xi) SEQ ID NO:15, SEQ ID NO:16, and SEQ ID NO:78; or

(xii) SEQ ID NO:15, SEQ ID NO:16, and SEQ ID NO:79.

In addition to the foregoing embodiments, provided herein are anti-CXCR3 antibodies comprising VH comprising an amino acid sequence of
SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, or SEQ ID NO:90.

In addition to the foregoing embodiments, provided herein are anti-CXCR3 antibodies comprising VH comprising an amino acid sequence of
SEQ ID NO:91, SEQ ID NO:92, SEQ ID NO:93, SEQ ID NO:94, SEQ ID NO:95, SEQ ID NO: 96, SEQ ID NO:97, SEQ ID NO:98, SEQ ID NO:99, SEQ ID NO:100, or SEQ ID NO:101.

In addition to the foregoing embodiments, provided herein are anti-CXCR3 antibodies comprising VH comprising an amino acid sequence selected of
SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, or SEQ ID NO:111.

In addition to the foregoing embodiments, provided herein are anti-CXCR3 antibodies comprising VH comprising an amino acid sequence of
SEQ ID NO:112, SEQ ID NO:113, SEQ ID NO:114, SEQ ID NO:115, SEQ ID NO:116, (SEQ ID NO:117, SEQ ID NO:118, SEQ ID NO:119, SEQ ID NO:120, SEQ ID NO:121, or SEQ ID NO:122.

In addition to the foregoing embodiments, provided herein are anti-CXCR3 antibodies comprising VL comprising an amino acid sequence of
SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, or SEQ ID NO:134.

In certain embodiments, the anti-human CXCR3 antibody comprises a VH/VL pair comprising the amino acid sequences shown in Table 3, respectively. (The indicated change(s) are with respect to the corresponding 53Hu37 VH or VL sequence (SEQ ID NOS:20, and 24), respectively):

TABLE 3

| VH | SEQ ID NO: | VL | SEQ ID NO: |
|---|---|---|---|
| G54K | 80 | S92R | 130 |
| G54K/N105F | 81 | 53Hu37 | 24 |
| G54K/T56K/N105F | 82 | S92R | 130 |
| G54K/T56K/N105F | 82 | 53Hu37 | 24 |
| G54R | 83 | N30R/S92R | 126 |
| G54R | 83 | S92R | 130 |
| G54R | 83 | 53Hu37 | 24 |
| G54R/N105F | 84 | N30R/S92R | 126 |
| G54R/N105F | 84 | 53Hu37 | 24 |
| G54R/N105W | 85 | 53Hu37 | 24 |
| G54R/T56R | 86 | N30R/S92R | 126 |
| G54R/T56R/N105F | 87 | N30R/S92R | 126 |
| G54R/T56R/N105F | 87 | T91R | 133 |
| G54R/T56R/N105F | 87 | 53Hu37 | 24 |
| H53R | 88 | 53Hu37 | 24 |
| H53R/N105Q | 89 | 53Hu37 | 24 |
| H53S | 90 | N30R/S92R | 126 |
| N105F | 91 | N30R/S92R | 126 |
| N105F | 91 | S92R | 130 |
| N105W | 92 | S92R | 130 |
| N105W | 92 | 53Hu37 | 24 |
| S103A | 93 | N30R/S92R | 126 |
| S103C | 94 | N30R/S92R | 126 |
| S103D | 95 | N30R/S92R | 126 |
| S103E | 96 | N30R/S92R | 126 |
| S103F | 97 | N30R/S92R | 126 |
| S103G | 98 | N30R/S92R | 126 |
| S103H | 99 | N30R/S92R | 126 |
| S103I | 100 | N30R/S92R | 126 |
| S103K | 101 | N30R/S92R | 126 |
| S103L | 102 | N30R/S92R | 126 |
| S103M | 103 | N30R/S92R | 126 |
| S103N | 104 | N30R/S92R | 126 |
| S103P | 105 | N30R/S92R | 126 |
| S103Q | 106 | N30R/S92R | 126 |
| S103R | 107 | N30R/S92R | 126 |
| S103T | 108 | N30R/S92R | 126 |
| S103V | 109 | N30R/S92R | 126 |
| S103W | 110 | N30R/S92R | 126 |
| S103Y | 111 | N30R/S92R | 126 |
| T30R | 112 | N30R/S92R | 126 |
| T50R | 121 | 53Hu37 | 24 |
| T56K | 113 | S92R | 130 |
| T56K | 113 | 53Hu37 | 24 |
| T56K/N105W | 114 | S92R | 130 |
| T56R | 115 | N30R/S92R | 126 |
| T56R | 115 | S92R | 130 |
| T56R | 115 | 53Hu37 | 24 |
| T56R/N105F | 116 | N30R/S92R | 126 |
| T56R/N105F | 116 | S92R | 130 |
| T56R/N105F | 116 | 53Hu37 | 24 |
| T56W | 117 | N30R/S92R | 126 |
| T56Y | 118 | N30R/S92R | 126 |
| 53Hu37 | 20 | N30I | 123 |
| 53Hu37 | 20 | N30K | 124 |
| 53Hu37 | 20 | N30R | 125 |
| 53Hu37 | 20 | N30R/S92R | 126 |
| 53Hu37 | 20 | N30S | 127 |
| 53Hu37 | 20 | N30W | 128 |
| 53Hu37 | 20 | N30Y | 129 |
| 53Hu37 | 20 | S92R | 130 |
| 53Hu37 | 20 | S93K | 131 |
| 53Hu37 | 20 | T91K | 132 |
| 53Hu37 | 20 | T91R | 133 |
| 53Hu37 | 20 | T91Y | 134 |
| Y57K | 119 | N30R/S92R | 126 |
| Y59W | 122 | N30R/S92R | 126 |
| Y102H | 120 | S92R | 130 |

### Neutralizing Antibodies

In certain embodiments, the humanized anti-human CXCR3 antibodies provided herein are CXCR3 neutralizing antibodies. In certain exemplary embodiments, the CXCR3 antibodies have neutralizing activity in addition to enhanced effector function. The combined effects of CXCR3 neutralization and CXCR3⁺ cell depletion may be advantageous whenever it is desirable to reduce or eliminate CXCR3-mediated effects, e.g., recruitment of T cells.

A “CXCR3 neutralizing antibody” binds to CXCR3 and blocks the activity of the receptor, such as the typical physiological and genetic responses resulting from CXCR3 ligands binding to CXCR3. Neutralizing activity may be complete (100% neutralization) or partial, e.g., approximately 10, 20, 30, 40, 50, 60, 70, 80, 90, 95 (or any percentage there between) or more neutralizing and will depend on various factors known to the skilled artisan, such as antibody concentration, affinity, and epitope as well as the particular assay used to evaluate neutralizing activity. The neutralizing activity of a CXCR3 neutralizing antibody may be shown by assays to measure inhibition of, e.g., ligand binding, GTP binding, calcium mobilization, cell chemotaxis, and/or receptor internalization. Numerous assays for determining the activity of neutralizing antibodies, and particularly CXCR3 neutralizing antibody, are known to the skilled artisan and may be readily adapted to verify that a particular antibody is neutralizing.

For example, in some embodiments, the neutralizing activity of an anti-CXCR3 antibody may be assessed by a chemotaxis assay, substantially as set forth in the package insert for the antibody produced by clone 49801 and sold by R&D Systems® (Cat. No. MAB160). The Neutralization Dose-50 (ND₅₀) is defined as the concentration of antibody required to yield one-half maximal inhibition of the cell surface CXCR3-mediated recombinant human CXCL11 (rhCXCL11) response in a responsive cell line, at a specific rhCXCL11 concentration. To measure the ability of the antibody to block rhCXCL11 induced chemotaxis of hCXCR3 transfected BaF/3 cells, rhCXCL11 at 7 ng/mL is added to the lower compartment of a 96-well chemotaxis chamber (NeuroProbe, Cabin John, Md.). The chemotaxis chamber is then assembled using a PVP-free polycarbonate filter (5 μm pore size). Serial dilutions of the antibody (e.g., from 0.001 to 10000 μg/mL) and 0.25×10⁶ cells/well are added to the top wells of the chamber. After incubation for 3 hours at 37° C. in a 5% CO₂-humidified incubator, the chamber is disassembled and the cells that migrate through to the lower chamber are transferred to a working plate and quantitated using, for example, Resazurin Fluorescence.

Colvin et al., Mol Cell Biol 26: 5838-49 (2006) describe additional assays that can be used, in certain embodiments, to determine the neutralizing activity of neutralizing anti-CXCR3 antibodies. Briefly, 300-19 cells, a murine pre-B-cell leukemia cell line that functionally expresses CXCR4, may be used. Following transfection, this line can functionally express other chemokine receptors, e.g., human CXCR3 (see, e.g., paragraphs 201-209 of U.S. Patent Application Publication No. 2010/0061983, which are incorporated herein by reference). 300-19 cells expressing human CXCR3 may be grown in complete RPMI medium containing 10% fetal bovine serum (FBS). To assess binding of CXCR3 ligands to CXCR3 in the presence of candidate neutralizing CXCR3 antibodies, 400,000 CXCR3/300-19 cells are placed into 96-well tissue culture plates in a total volume of 150 μL of binding buffer (0.5% BSA, 5 mM MgCl₂, 1 mM CaCl₂, 50 mM HEPES, pH 7.4). A total of 0.04 nM of¹²⁵1 labeled CXCL10 (New England Nuclear, Boston, Mass.) or CXCL11 (Amersham Biosciences Piscataway, N.J.) and 5×10⁶ nM to 500 nM of unlabeled CXCL10 or CXCL11 (Peprotech, Rocky Hill, N.J.) may be added to the cells and incubated for 90 min at room temperature with shaking. The cells are transferred onto 96-well filter plates (Millipore, Billerica, Mass.) that are presoaked in 0.3% polyethyleneimine and washed three times with 200 μL binding buffer supplemented with 0.5 M NaCl. The plates are dried, and the radioactivity is measured after the addition of scintillation fluid in a Wallac Microbeta scintillation counter (Perkin-Elmer Life Sciences, Boston, Mass.). Binding of CXCL9 may be assessed analogously to CXCL10 and CXCL11.

In certain embodiments, the antibodies disclosed herein can prevent or reduce calcium flux into CXCR3-expressing cells. In some embodiments, calcium flux may be detected in cells such as CXCR3/300-19 cells. Approximately 5×10⁶ cells are suspended in 2 mL of RPMI medium with 1% bovine serum albumin (BSA). Fifteen micrograms of Fura-2 (Molecular Probes, Eugene, Oreg.) are added and the cells are incubated at 37° C. for 20 min. The cells are washed twice in PBS and resuspended in 2 mL of calcium flux buffer (145 mM NaCl, 4 mM KCl, 1 mM NaHPO₄, 1.8 mM CaCl₂, 25 mM HEPES, 0.8 mM MgCl₂, and 22 mM glucose). Fluorescence readings are measured at 37° C. in a DeltaRAM fluorimeter (Photon Technology International, Lawrenceville, N.J.). Before and after the addition of chemokines (e.g., CXCL9, CXCL10, or CXCL11), intracellular calcium concentrations are recorded as the excitation fluorescence intensity emitted at 510 nm in response to sequential excitation at 340 nm and 380 nm and presented as the relative ratio of fluorescence at 340 nm to that at 380 nm.

In certain embodiments, CXCR3 neutralization can be evaluated by measuring a reduction in receptor internalization. In some embodiments, receptor internalization assays may be performed by incubating about 2.5×10⁵ cells, such as CXCR3/300-19 cells, in RPMI medium with 1% BSA with various concentrations of CXCL10, CXCL11, or CXCL9 for 30 min at 37° C. The cells may then be washed with ice-cold flow cytometry staining buffer and subsequently analyzed for surface expression of CXCR3 using a PE-conjugated CXCR3 antibody.

As assessed by any of the above assays, a neutralizing anti-CXCR3 antibody may have, in certain embodiments, an ND₅₀ of approximately 0.01, 0.02, 0.05, 0.1, 0.2, 0.5, 1, 2, 3, 4, 5, 6, 7, 8, 9, 10, 15, 20, 40, 50, or 100 μg/mL. In particular embodiments, the ND₅₀ may be 0.5-12 μg/mL, and in more particular embodiments, 1-6 μg/mL.

Inhibition of cell migration, recruitment, or accumulation by an antibody or antigen-binding fragment provided herein can be assessed by any method known to those skilled in the art. Such methods can include, for example, analysis of biopsies by immunohistochemistry, flow cytometry, RT-PCR, etc., to assess the number of cells, such as CXCR3⁺ cells, in one or more population of cells or one or more locations within the body or within an organ. Cell migration, recruitment, or accumulation can be inhibited by at least or about 10%, 15%, 20%, 25%, 30%, 35%, 40%, 45%, 50%, 55%, 60%, 65%, 70%, 75%, 80%, 85%, 90%, 95%, or more compared to the migration, recruitment, or accumulation in the absence of an antibody or antigen-binding fragment provided herein.

### Nucleotide Sequences

Also provided herein are nucleotide sequences encoding the amino acid sequences disclosed herein. In some embodiments, a nucleotide sequence encodes an antibody or fragment capable of depleting CXCR3⁺ cells in vitro and/or in vivo. In certain embodiments, the nucleotide sequences can be used to prepare expression vectors for the expression of anti-CXCR3 antibodies or antigen-binding fragments thereof in cells (e.g., expression in mammalian cells).

Also disclosed herein, in certain embodiments, are polynucleotides substantially identical to those coding for the amino acid sequences disclosed herein. Substantially identical sequences may be polymorphic sequences, i.e., alternative sequences or alleles in a population. Substantially identical sequences may also comprise mutagenized sequences, including sequences comprising silent mutations. A mutation may comprise one or more nucleotide residue changes, a deletion of one or more nucleotide residues, or an insertion of one or more additional nucleotide residues. Substantially identical sequences may also comprise various nucleotide sequences that encode for the same amino acid at any given amino acid position in an amino acid sequence disclosed herein, due to the degeneracy of the nucleic acid code. Also included within substantially identical sequences are sequences that encode a chain or chains of an antibody that retains the ability to deplete CXCR3⁺ cells in vitro and/or in vivo.

In certain embodiments, a nucleic acid provided herein encodes the amino acid sequence of a chain or chains in an antibody or fragment capable of depleting CXCR3-expressing cells provided herein, or the nucleic acid may hybridize under stringent conditions to a nucleic acid that encodes the amino acid sequence of a chain or chains in the antibody or antigen-binding fragment thereof.

In certain embodiments, a polynucleotide sequence is disclosed herein, comprising a nucleotide sequence encoding an amino acid sequence of a VH domain of an anti-CXCR3 antibody or antigen-binding fragment thereof, and which is at least about 80-100%, (e.g., about 80%, 85%, 90%, 91%, 92%, 93%, 94%, 95%, 96%, 97%, 98%, 99%, or 100%) identical (or any percentage therebetween) to the nucleotide sequence encoding the heavy chain of the antibody. In certain embodiments, the polynucleotide sequence may comprise a nucleotide sequence having 0, 1, 2, 3, 4, 5, 6, 7, 8, 9, or 10 mutations (including additions, deletions, and substitutions, such as conservative substitutions) relative to the nucleotide sequence encoding the heavy chain of the antibody.

In certain embodiments, a polynucleotide sequence is disclosed herein, comprising a nucleotide sequence encoding an amino acid sequence of a VL domain of an anti-CXCR3 antibody or fragment, and which is at least about 80-100%, (e.g., about 80%, 85%, 90%, 91%, 92%, 93%, 94%, 95%, 96%, 97%, 98%, 99%, or 100%) identical (or any percentage therebetween) to the nucleotide sequence encoding the light chain of the antibody. In certain embodiments, the polynucleotide sequence may comprise a nucleotide sequence having 0, 1, 2, 3, 4, 5, 6, 7, 8, 9, or 10 mutations (including additions, deletions, and substitutions, such as conservative substitutions) relative to the nucleotide sequence encoding the light chain of the antibody.

In particular embodiments, a polynucleotide sequence is disclosed herein, comprising a nucleotide sequence that is at least about 80%, 85%, 90%, 91% 92%, 93%, 94%, 95%, 96%, 97%, 98%, 99%, or 100% identical (or any percentage in between) to a VH amino acid sequence and at least about 80%, 85%, 90%, 91%, 92%, 93%, 94%, 95%, 96%, 97%, 98%, 99%, or 100% identical (or any percentage in between) to a VL amino acid sequence, where the nucleotide sequences encode the heavy and light chain amino acid sequences of any of the antibodies disclosed herein.

The disclosed polynucleotides may be obtained by any method known in the art. For example, if the nucleotide sequence of an antibody is known, a polynucleotide encoding the antibody may be assembled from chemically synthesized oligonucleotides. This could involve, for example, the synthesis of overlapping oligonucleotides containing portions of the sequence encoding the antibody, annealing and ligating those oligonucleotides, and then amplifying the ligated oligonucleotides by PCR. The disclosed polynucleotides can also be generated from any other suitable source of nucleic acids, such as an antibody cDNA library, or a cDNA library isolated from any tissue or cells expressing the antibody (e.g., from hybridoma cells selected to express an antibody).

### Expression of Anti-CXCR3 Antibodies or Antigen-Binding Fragments Thereof

Following manipulation of the nuclei acid encoding the humanized anti-CXCR3 antibodies or antigen-binding fragments thereof provided herein, the encoding nucleic acid is typically inserted in an expression vector for introduction into host cells that may be used to produce the desired quantity of the encoded antibodies, or antigen-binding fragments thereof. Suitable vectors for expression are known in the art. Suitable host cells include, e.g., CHO, COS, Sf9, and/or other human or nonhuman cell lines. In some embodiments, the host cells transiently or stably express the nucleic acid on the vector when cultured in culture medium, thereby providing a method for producing the antibodies or fragments disclosed herein.

The term “vector” or “expression vector” is used herein to describe a vehicle for introducing into and expressing a desired gene in a cell. As known to those skilled in the art, such vectors include, for example, plasmids, phages, viruses and retroviruses. In general, suitable vectors can comprise a selection marker, appropriate restriction sites to facilitate cloning of the desired gene and the ability to enter and/or replicate in eukaryotic or prokaryotic cells.

Numerous expression vector systems may be employed for the purposes of expressing the anti-CXCR3 antibodies provided herein. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals. In some embodiments the cloned variable region genes are inserted into an expression vector along with the heavy and light chain constant region genes as discussed above. In some embodiments the heavy and light chain constant regions are human.

In other embodiments, the anti-CXCR3 antibodies, or antigen-binding fragments thereof provided herein may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides provided herein in eukaryotic host cells. Compatible IRES sequences are disclosed in U.S. Pat. No. 6,193,980, which is incorporated by reference herein. Those skilled in the art will appreciate that such expression systems may be used to effectively produce the full range of polypeptides disclosed in the instant application.

Once a vector or DNA sequence encoding an antibody, or antigen-binding fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). In some embodiments, plasmid introduction into the host is via electroporation. The transformed cells are grown under conditions appropriate to the production of the encoded amino acid sequence, for example, antibody light chains and heavy chains, and assayed for the production of the encoded amino acid sequence. Exemplary assay techniques include enzyme-linked immunosorbent assay (ELISA), radioimmunoassay (RIA), flow cytometry, immunohistochemistry and the like.

“Host cells” refers to cells into which vectors constructed using recombinant nucleic acid techniques and encoding at least one heterologous protein have been introduced. In descriptions of processes for isolation of polypeptides from recombinant hosts, the terms “cell” and “cell culture” are used interchangeably to denote the source of the encoded protein, e.g., antibody or antigen-binding fragment thereof, unless it is clearly specified otherwise. In other words, recovery of polypeptide from the “cells” may mean either from spun down whole cells, or from the cell culture containing both the medium and the suspended cells.

In one embodiment, the host cell line used for antibody expression is of mammalian origin; those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), SP2/0 (mouse myeloma), BFA-1c1BPT (bovine endothelial cells), RAJI (human lymphocyte), 293 (human kidney). In one embodiment NSO cells may be used. In some embodiments, CHO cells are used. Host cell lines are typically available from commercial services, the American Tissue Culture Collection, or from published literature.

In one embodiment, the cell line provides for altered glycosylation, e.g., afucosylation, of the antibody expressed therefrom (e.g., PER.C6® (Crucell) or FUT8-knock-out CHO cell lines (Potelligent® Cells) (Biowa, Princeton, N.J.)). Alternatively, the cell may be deficient in one or more glycosidases required for early stage processing of N-glycans and/or the culture conditions may be such that the activity of one or more of these glycosidases is inhibited. For example, the cell may be deficient in one or more glycosidases such as alpha-glucosidase I, alpha-glucosidase II, and alpha-mannosidase I. In addition, or alternatively, the engineered cell may be contacted with an inhibitor of one or more glycosidases such as alpha-glucosidase I, alpha-glucosidase II, and alpha-mannosidase I. In certain embodiments, the inhibitor is an inhibitor of alpha-mannosidase I, e.g., the alpha-mannosidase I specific inhibitor, kifunensine. Exemplary methods for culturing host cells with kifunensine and other inhibitors are disclosed in U.S. Pat. No. 8,071,336, which is incorporated by reference herein in its entirety. In certain embodiments, kifunensine treatment results in antibodies having at least 50% Man₅₋₉(GlcNAc)₂ N-glycans, wherein Man8 and Man9-containing N-glycans together are the major species.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Nucleic acid encoding the anti-CXCR3 antibodies, or fragments thereof, provided herein can also be expressed in non-mammalian cells such as bacteria or yeast or plant cells. In this regard it will be appreciated that various unicellular non-mammalian microorganisms such as bacteria can also be used to express the antibodies and antigen-binding fragments thereof provided herein; i.e. those capable of being grown in cultures or fermentation. Suitable bacteria include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides must be isolated, purified and then assembled into functional molecules.

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the Trpl lesion as a characteristic of the yeast host cell genome then provides an effective environment for detecting transformation by growth in the absence of tryptophan.

### Pharmaceutical Formulations and Methods of Administration

Provided herein are pharmaceutical compositions comprising a humanized anti-human CXCR3 antibody herein disclosed, and a pharmaceutically acceptable carrier.

Methods of preparing and administering antibodies or antigen-binding fragments thereof provided herein to a subject are well known to or are readily determined by those skilled in the art. The route of administration of the antibodies, or fragments thereof, be oral, parenteral (such as intravenous, intramuscular, intraperitoneal, or subcutaneous), by inhalation or topical. In some embodiments, the antibodies provided herein are formulated for intravenous administration. In some embodiments a suitable pharmaceutical composition for injection comprises a buffer (e.g. acetate, phosphate or citrate buffer), a surfactant (e.g. polysorbate), optionally a stabilizer agent (e.g. human albumin), etc.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will preferably have labels or package inserts indicating that the associated compositions are useful for treating a subject suffering from, or predisposed to autoimmune or neoplastic disorders.

Doses of the antibodies or antigen-binding fragments thereof provided herein for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated.

In some embodiments the dose may range, e.g., from about 0.0001 to 100 mg/kg, or 0.01 to 5 mg/kg of the host body weight.

Subjects can be administered such doses daily, on alternative days, weekly or according to any other schedule determined by empirical analysis. Antibodies, or antigen-binding fragments thereof provided herein can be administered on multiple occasions. Intervals between single dosages can be, e.g., daily, weekly, monthly or yearly. Intervals can also be irregular as indicated by measuring blood levels of polypeptide or target molecule in the patient.

Antibodies or antigen-binding fragments thereof provided herein can optionally be administered in combination with other agents that are used in treating the disorder or condition in need of treatment (e.g., prophylactic or therapeutic). Preferred additional agents are those which are art recognized and are standardly administered for a particular disorder.

### Methods of Treating CXCR3-Associated Disease or Disorders

The CXCR3 antibodies, or antigen-binding fragments thereof provided herein are useful for antagonizing CXCR3 activity. In some embodiments, the antibodies and antigen-binding fragments are used in methods to inhibit CXCR3 binding to one or more ligands, such as CXCL9, CXCL10, and/or CXCL11; inhibit migration, accumulation, recruitment, or infiltration of CXCR3⁺ cells, such as to a site of inflammation; and/or deplete CXCR3⁺ cells. In some embodiments, the antibodies and antigen-binding fragments are used in methods to deplete CXCR3⁺ cells in vivo. CXCR3⁺ cells include, but are not limited to, CXCR3⁺/CD4⁺ T cell, CXCR3⁺/CD8⁺ T cell, and CXCR3⁺/CD19⁺ B cell subsets.

In certain embodiments, methods are provided for treating CXCR3-associated diseases or disorders by administering to a subject in need of thereof a pharmaceutical composition comprising one or more CXCR3 antibody, or antigen-binding fragment thereof. In one embodiment, a method of treating or reducing the progression of a T-cell-mediated autoimmune disease is provided. The method includes the step of administering to a subject in need thereof a humanized anti-human CXCR3 antibody or antigen-binding fragment thereof disclosed herein, thereby treating or reducing the progression of the T-cell-mediated autoimmune disease. In certain embodiments, the T-cell-mediated autoimmune disease is vitiligo. In some embodiments, a subject in need thereof includes subject who have been diagnosed with a CXCR3-associated disease or a T-cell-mediated autoimmune disease or is predisposed to develop a CXCR3-associated disease or a T-cell-mediated autoimmune disease as described herein.

Subjects to be treated by the methods provided herein can include humans or other mammals. In a one embodiment, the subject is a human. In various embodiments, a subject can be treated prophylactically or after onset of any condition associated with aberrant CXCR3 activity or any condition in which the disruption of CXCR3 signaling could be therapeutically beneficial.

In some embodiments, a subject can be treated prophylactically or after onset of vitiligo.

A “subject having vitiligo” is any subject who has patchy reduced skin pigmentation, usually of autoimmune or unknown cause, that is consistent with a medical diagnosis of vitiligo. Affected areas can include face, hands, feet, arms, legs, lips, or any combination thereof, but other areas, e.g., neck or trunk, may be involved. In some instances, there is reduced hair pigmentation.

### EXAMPLES

The antibodies, compositions of matter, and methods are further illustrated by the following examples which should not be construed as further limiting. The contents of Sequence Listing, figures and all references, patents and published patent applications cited throughout this application are expressly incorporated herein by reference.

### Example 1: Generation of Humanized Anti-CXCR3 Monoclonal Antibodies

Anti-CXCR3 monoclonal antibodies were generated as described in WO2013/109974. Humanized clone 53 monoclonal antibodies were generated as described below.

Humanized versions of clone 53 were made that are capable of directing depletion of CXCR3-expressing cells. Humanized clone 53 monoclonal antibodies were generated having the VH and VK sequences as shown in Table 4.

Germinality index scores for each of the humanized variants shown in Table 4. Heavy chains are compared to IGHV3-23*03/IGHJ4*03 germline sequences; light chains are compared to IGKV1-9*01/IGKJ2*01 germline sequences.

TABLE 4

| | VH SEQ | VL SEQ | Germinality | Germinality |
|---|---|---|---|---|
| HuAntibody | ID NO | ID NO | Index 1^{a} | Index 2^{b} |
| 23 | 18 | 21 | 0.885 | 0.950 |
| 24 | 19 | 21 | 0.872 | 0.933 |
| 25 | 20 | 21 | 0.877 | 0.939 |
| 26 | 18 | 22 | 0.890 | 0.955 |
| 27 | 19 | 22 | 0.877 | 0.939 |
| 29 | 20 | 22 | 0.881 | 0.944 |
| 30 | 18 | 23 | 0.890 | 0.955 |
| 31 | 19 | 23 | 0.877 | 0.939 |
| 33 | 20 | 23 | 0.881 | 0.944 |
| 34 | 18 | 24 | 0.895 | 0.961 |
| 35 | 19 | 24 | 0.881 | 0.944 |
| 37 | 20 | 24 | 0.886 | 0.950 |

^{a}Germinality Index (1)
Heavy: Pairwise comparison of all residues except those contributed by D-region
Light: Pairwise comparison across all residues
^{b}Germinality Index (2)
Heavy: Pairwise comparison of all framework residues only (as delimited by IMTG definitions)
Light: Pairwise comparison of all framework residues only (as delimited by IMTG definitions)

Binding characteristics for each of the humanized monoclonal antibodies provided herein are shown in Table 5.

TABLE 5
Binding Kinetics

| HuAntibody | ka (1/Ms) | kd (1/s) | KD (M) |
|---|---|---|---|
| 23 | 7.56E+04 | 2.45E−05 | 3.22E−10 |
| 24 | 1.01E+05 | 6.16E−06 | 6.14E−11 |
| 25 | 9.95E+04 | 6.21E−05 | 6.23E−10 |
| 26 | 1.01E+05 | 5.46E−05 | 5.37E−10 |
| 27 | 1.13E+05 | 8.34E−06 | 7.37E−11 |
| 29 | 1.15E+05 | 3.50E−05 | 3.03E−10 |
| 30 | 9.37E+04 | 1.26E−04 | 1.34E−09 |
| 31 | 1.11E+05 | 5.00E−05 | 4.49E−10 |
| 33 | 9.66E+04 | 6.35E−05 | 6.56E−10 |
| 34 | 8.89E+04 | 9.19E−05 | 1.03E−09 |
| 35 | 1.02E+05 | 4.73E−06 | 4.56E−11 |
| 37 | 6.74E+04 | 6.48E−05 | 9.57E−10 |

Table 6 shows the binding characteristics and germinality for clone 53 (53), chimeric clone 53 (Ch53) and humanized versions of clone 53 (53Hu1-53Hu20)

TABLE 6

| | | | | Germinality | Germinality |
|---|---|---|---|---|---|
| Antibody | ka (1/Ms) | kd (1/s) | KD (M) | Index 1^{a} | Index 2^{b} |
| 53 | 7.96E+04 | 5.81E−04 | 7.31E−09 | — | — |
| 53Ch | 5.61E+04 | 7.83E−05 | 1.60E−09 | — | — |
| 53Hu01 | poor binding | poor | poor binding | 0.904 | 0.972 |
| 53Hu02 | 3.53E+04 | 1.50E−04 | 4.26E−09 | 0.896 | 0.922 |
| 53Hu03 | 6.88E+04 | 1.13E−04 | 1.72E−09 | 0.827 | 0.878 |
| 53Hu04 | 2.54E+04 | 1.93E−04 | 8.05E−09 | 0.876 | 0.938 |
| 53Hu05 | no binding | no binding | no binding | 0.891 | 0.956 |
| 53Hu06 | 7.01E+04 | 1.25E−08 | 1.81E−13 | 0.867 | 0.928 |
| 53Hu07 | poor binding | poor binding | poor binding | 0.867 | 0.928 |
| 53Hu08 | 6.02E+04 | 1.90E−07 | 3.07E−12 | 0.867 | 0.928 |
| 53Hu09 | poor binding | poor binding | poor binding | 0.867 | 0.928 |
| 53Hu10 | 6.74E+04 | 3.17E−05 | 4.72E−10 | 0.867 | 0.928 |
| 53Hu11 | 3.33E+04 | 3.50E−05 | 1.10E−09 | 0.867 | 0.928 |
| 53Hu12 | no binding | no binding | no binding | 0.867 | 0.927 |
| 53Hu13 | no protein | no binding | no protein | 0.867 | 0.927 |
| 53Hu14 | 6.42E+04 | 1.01E−07 | 1.62E−12 | 0.867 | 0.927 |
| 53Hu15 | 5.12E+04 | 1.48E−07 | 2.99E−10 | 0.881 | 0.944 |
| 53Hu16 | 5.77E+04 | 7.32E−05 | 1.29E−09 | 0.881 | 0.944 |
| 53Hu17 | 6.89 + 04 | 1.18E−07 | 1.76E−12 | 0.872 | 0.933 |
| 53Hu18 | 7.73E+04 | 3.21E−06 | 4.04E−11 | 0.872 | 0.933 |

^{a}Germinality Index (1)
Heavy: Pairwise comparison of all residues except those contributed by D-region
Light: Pairwise comparison across all residues
^{b}Germinality Index (2)
Heavy: Pairwise comparison of all framework residues only (as delimited by IMTG definitions)
Light: Pairwise comparison of all framework residues only (as delimited by IMTG definitions)

As indicated by the above data, humanized antibodies provided herein have significantly improved binding characteristics and while having a favorable germinality index.

### Example 2: CDR Optimization

A number of VH CDR and/or VL CDR variants were made. Binding avidity to recombinant human CXCR3 was measured using Biacore. Mutants with binding at least as strong as for 53Hu37 are shown in Table 3.

### Example 3: CXCR3-173 is a Depleting Antibody

The hamster anti-mouse CXCR3 monoclonal (clone CXCR3-173) was used as a surrogate antibody in pre-clinical experiments. CXCR3-173 has previously been described as a blocking antibody that does not deplete CD4+ T cells in vivo. (see Uppaluri et al., Transplantation 86: 137-47 (2008)).

The following Fc variants of the hamster CXCR3-173 mAb were prepared to test the effector function of the antibody: aglycosylated N297G variant of mouse IgG1 (CXCR3 mIgG1 agly); wild type mouse IgG2a chimera (CXCR3 mIgG2a WT); mouse IgG2a chimera modified to abrogate depleting capability (CXCR3 mIgG2a Dab); and wild type mouse IgG3 (CXCR3 mIgG3).

Eight- to ten-week-old C57BL/6 mice (Jackson Laboratories, n=5 per group) were administered a single 5 mg/kg intravenous dose of antibody and then blood was harvested 24 h later for flow cytometry analysis using the following markers: CD4 and CD8 T cells by TCRab, CD4, and CD8; memory CD4 and CD8 T cells by TCRab, CD4, CD8, CD62L and CD44. Cells were quantitated using Count Bright beads (Invitrogen) following the manufacturer's protocol to determine the total cells/microliter of blood.

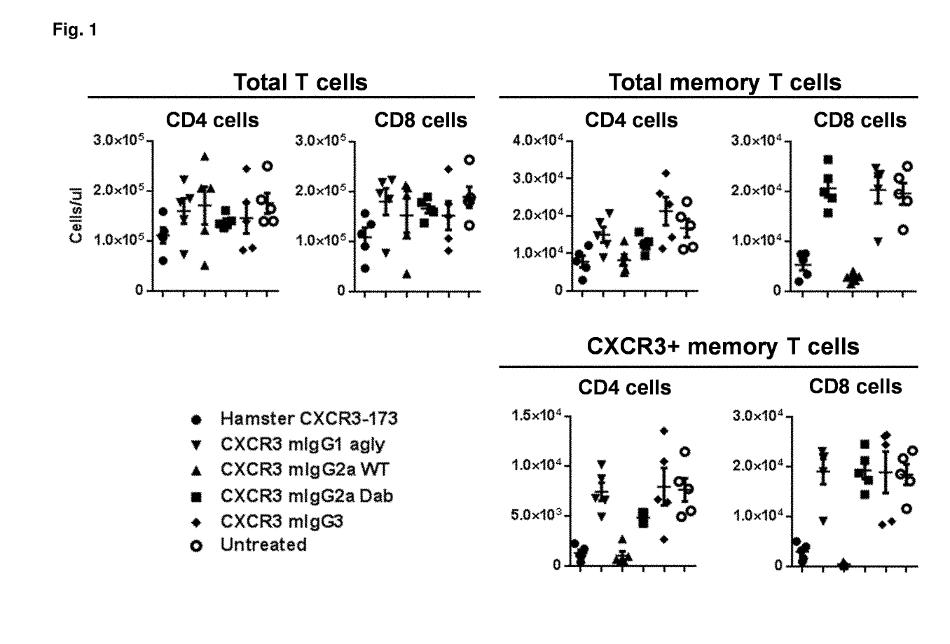

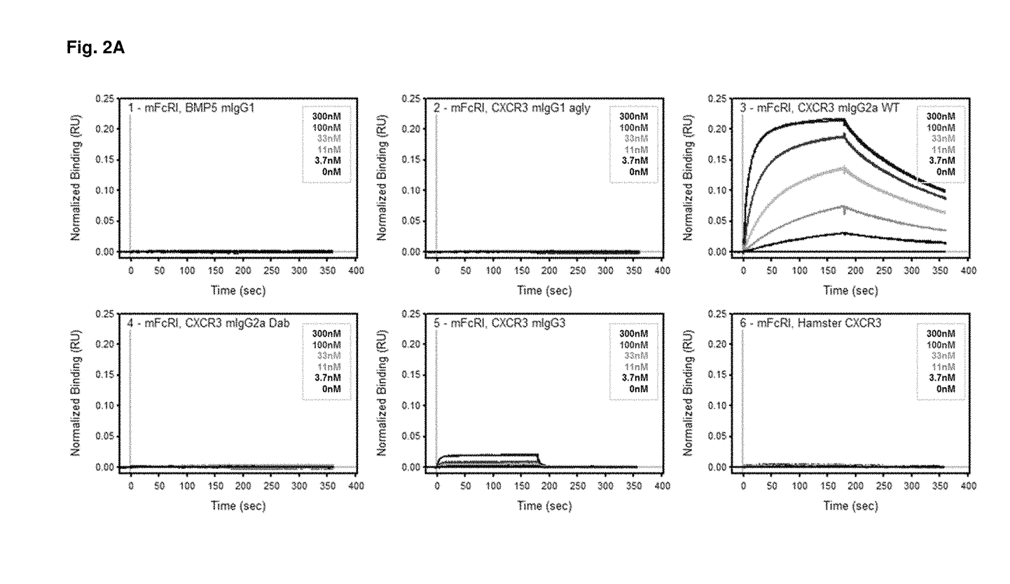

Results from gating on total T lymphocytes and subsets of T lymphocytes are shown in FIG. 1. As shown in the figure, it was surprisingly demonstrated for the first time that hamster CXCR3-173 depletes CD4+ and CD8+ memory T cells when administered to mice.

### Example 4: Effector Function of Fc Variants of CXCR3-173

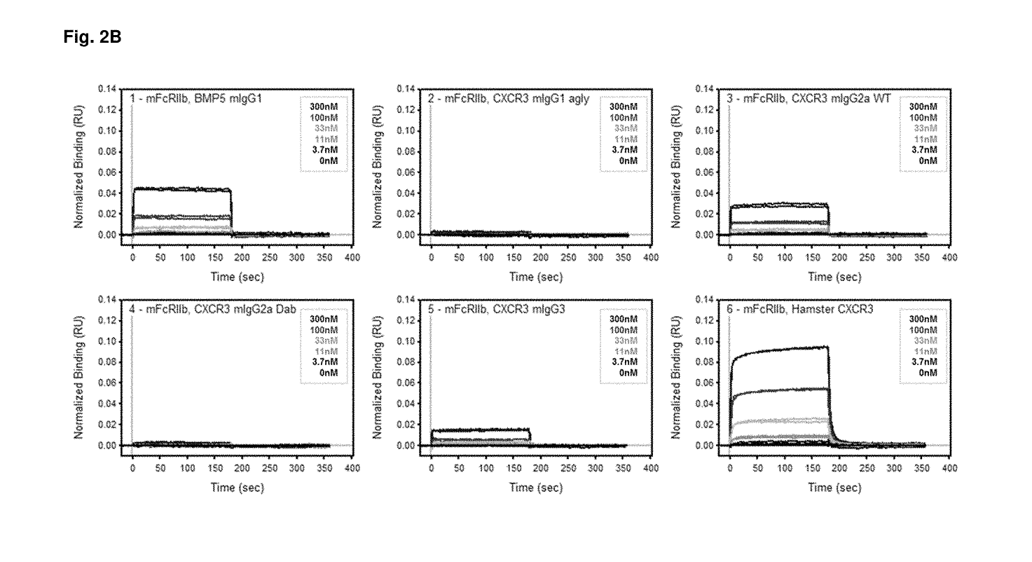

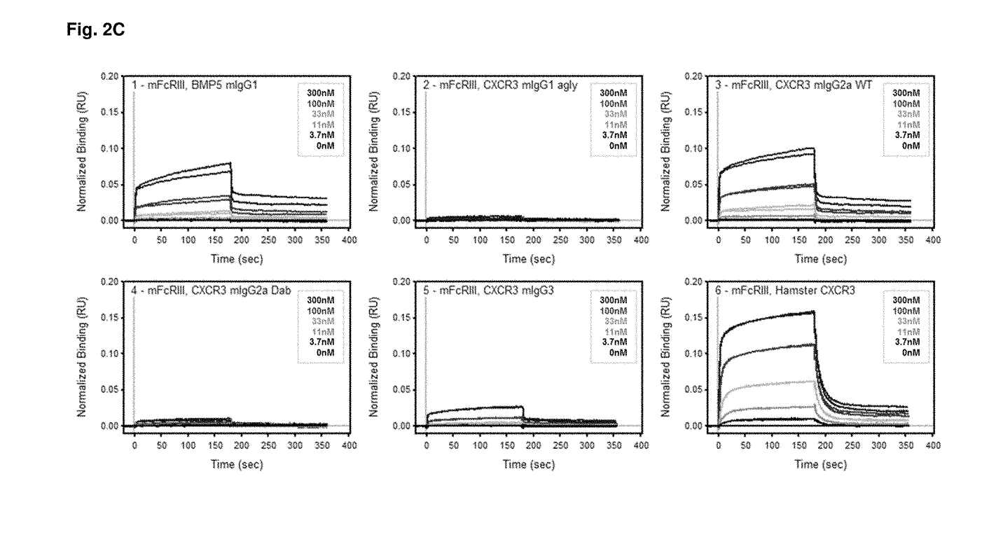

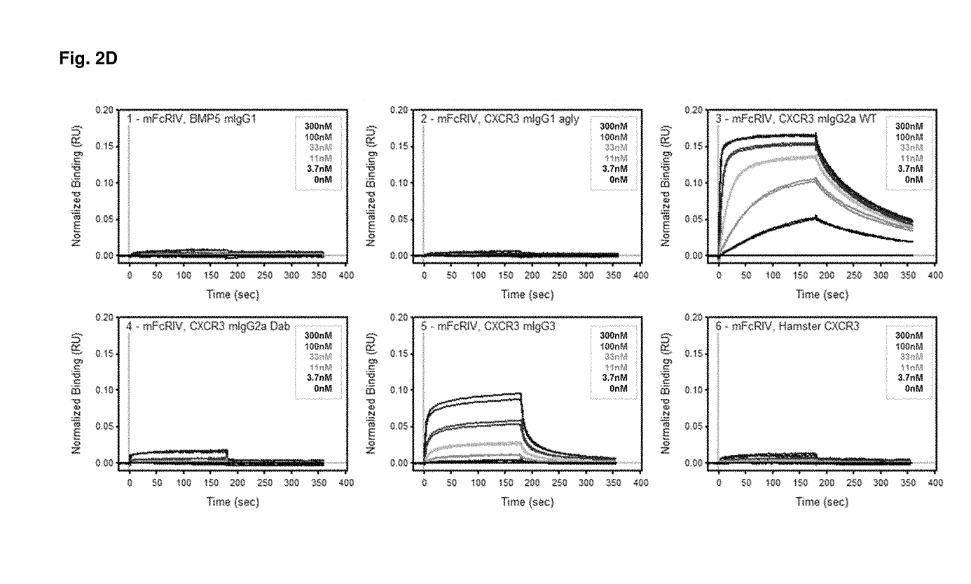

A Biacore 3000 instrument was used to assess mouse Fey receptor binding of the Fc-engineered versions of CXCR3-173 using an antibody capture approach. Recombinant protein A/G (Pierce) was covalently immobilized to a CMS sensor chip using amine chemistry. CXCR3-173 antibodies were diluted to 5 μm/mL in HBS-EP buffer and injected to the protein A/G chip for 30 sec at 10 μL/min flow. Recombinant mouse FcγRI (CD64), FeyRIIb (CD32), FcγRIII (CD16), and FcγIV (CD16-2) from R&D Systems were diluted 3-fold from 300 to 3.7 nM in HBS-EP buffer and injected in duplicate to the captured antibodies at 30 μL/min flow-rate. The surface was regenerated with glycine 2.0 (GE Healthcare). The binding response was normalized to the RU amount of protein A/G capture. Results are shown in FIGS. 2A-2D.

As shown in the figures, hamster CXCR3-173 modified to have wild type murine IgG2a isotype binds to all four recombinant mouse (rm) Fcγ receptors, although the dAB mutation significantly reduces this binding. CXCR3-173 modified to have wild type murine IgG3 isotype also binds to all four recombinant mouse Fcγ receptors. Original, unmodified hamster CXCR3-173 binds to rmFcγRIIb and rmFcγRIII better than the IgG2a isotype variant, and the aglycosylated mIgG1 isotype variant does not bind to any rmFcγR.

### Example 5: In Vitro Effector Function

Humanized anti-CXCR3 mAb and Fc-engineered versions thereof were studied in a series of ADCC assays. Fc-engineered versions of humanized anti-CXCR3 mAb were prepared using standard methods. Defucoylsated versions were prepared by culturing cells expressing the humanized mAb in the presence of kifunensine.

ADCC assays were performed using primary human NK cells or the NK9.2 cell line overexpressing CD16 having the valine polymorphism (Conkwest) as effector cells and using CHO transfected cells overexpressing human CXCR3 (A isoform) as target cells.

For the assay when primary NK cells were used as effectors, the NK cells were purified from a leucopak of a normal donor and cultured for 24 h in IL-2 then plated at a 5:1 E:T ratio with the CHO-human CXCR3 target cells that had been labeled overnight with chromium. The cultures were incubated for 3 hrs in the tissue culture incubator followed by washing and lysing with 1% Tritron-X before reading the supernatant on the gamma counter.

For the assay when NK9.2 cells were used as effectors, the NK9.2 cells were expanded for 2 weeks in IL-2 following the manufacturer's recommendations. On the day of the assay, the NK9.2 cells (70,000 cells) were labeled with calcein AM (Invitrogen) and incubated for 30 minutes with appropriately diluted antibodies to allow the antibody to bind to CXCR3 on the target cells. NK cells were plated at a 3:1 effector to target cell ratio and the cultures incubated for an hour in the tissue culture incubator. The cells were lysed with Triton X-100 at the end of the culture period and plates were read using M5 plate reader (492 nm excitation and 515 nm emission).

Human IgG1 (Sigma) was used as a negative control and lysis of CD52-overexpressing CHO cells treated with alemtuzumab (monoclonal anti-CD52 antibody) served as the positive control for lysis. The signal is expressed in arbitrary fluorescence units (AFU). Percent cytotoxicity is expressed by (experimental lysis−spontaneous lysis)/(maximal lysis−spontaneous lysis)×100%.

Humanized anti-CXCR3 mAb 53Hu37 having human a IgG1 Fc and Fc-engineered versions were tested in ADCC assays. The Fc-engineered versions M1 (S239D/D332E (EU notation), M2 (G236A/S267E/H268F/S324T/I332E (EU notation), “AEFTE”), and M3 (S239D/H268F/S324T/I332E (EU notation), “DFTE”) contain amino acid changes allowing for enhanced ADCC or CDC activity. A fourth Fc-engineered version was created by kifunensine treatment of the cell line making the wild-type antibody.

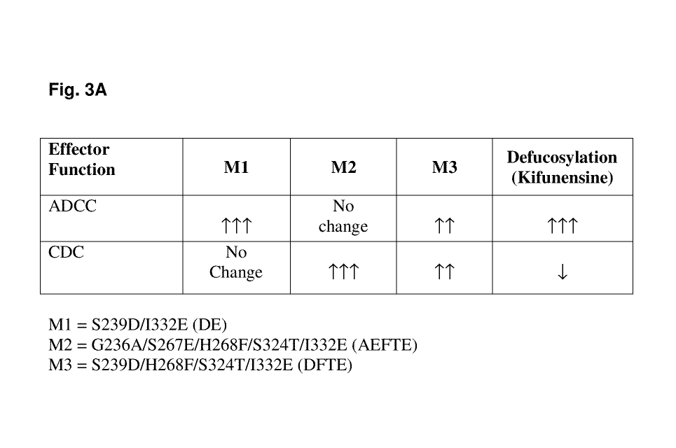

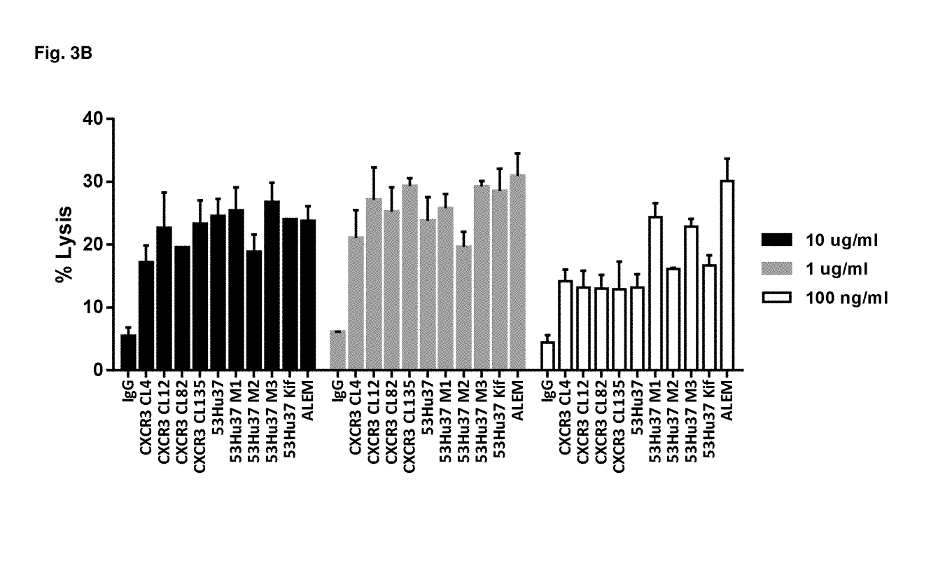

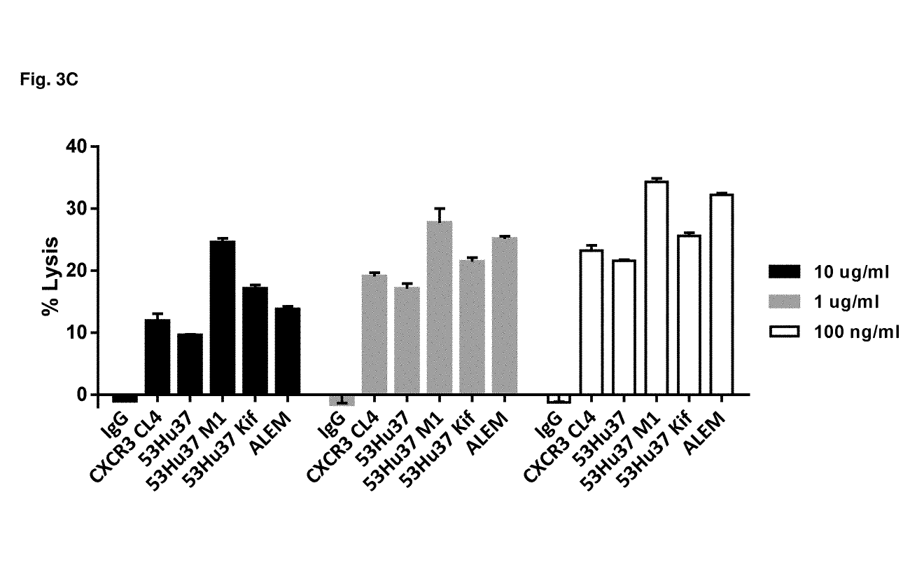

Anti-human CXCR3 clones clone 4 (CXCR3 CL4), clone 12 (CXCR3 CL12), clone 82 (CXCR3 CL82), clone 135 (CXCR3 CL135) were also tested. In independent experiments, assays were run with differing effector cells, effector:target (E:T) ratios, and concentrations of antibody. Representative results are shown in FIG. 3B and FIG. 3C.

FIG. 3A summarizes effector function of M1, M2, M3, and defucosylated versions of 53Hu37. FIG. 3B shows results of the assay using primary NK cells as effectors. FIG. 3C shows results of the assay using NK9.2 cells as effectors.

### Example 6: Fcγ Receptor Binding

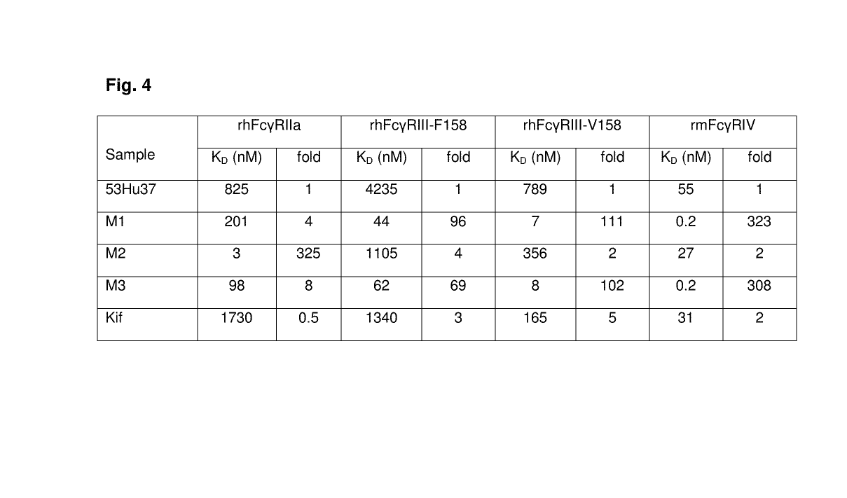

A Biacore T200 instrument was used to assess the human and mouse Fey receptor binding affinity of humanized anti-CXCR3 mAb 53Hu37 and Fc-engineered versions of 53Hu37. Protein A from Sigma was immobilized to a CMS series S chip using amine chemistry. The antibodies were injected into the protein A chip, and multiple concentrations of recombinant human and mouse Fey receptors (R&D Systems) were injected into the captured antibodies. A wide concentration range of receptors was used to span the low affinity binders and the high affinity binders (1.2 nM up to 5 μM). Each sample was injected in duplicate. The binding sensorgrams were fit to a 1:1 kinetic binding model. Quantitative results are summarized in FIG. 4.

As shown in FIG. 4, the M1 and M3 Fc-engineered versions had improved affinity to both hFcγRIII and mFcγRIV, M2 had increased binding to hFcγRIIa, and kifunensine-treated 53Hu37 displayed moderate increases in hFcγRIII and mFcγRIV compared to the Fc-engineered versions.

### Example 7

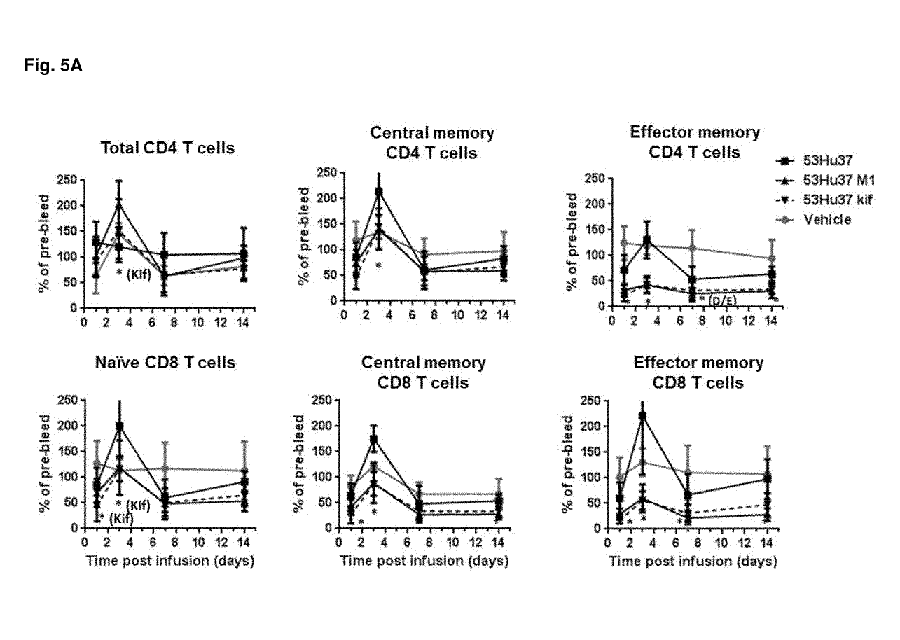

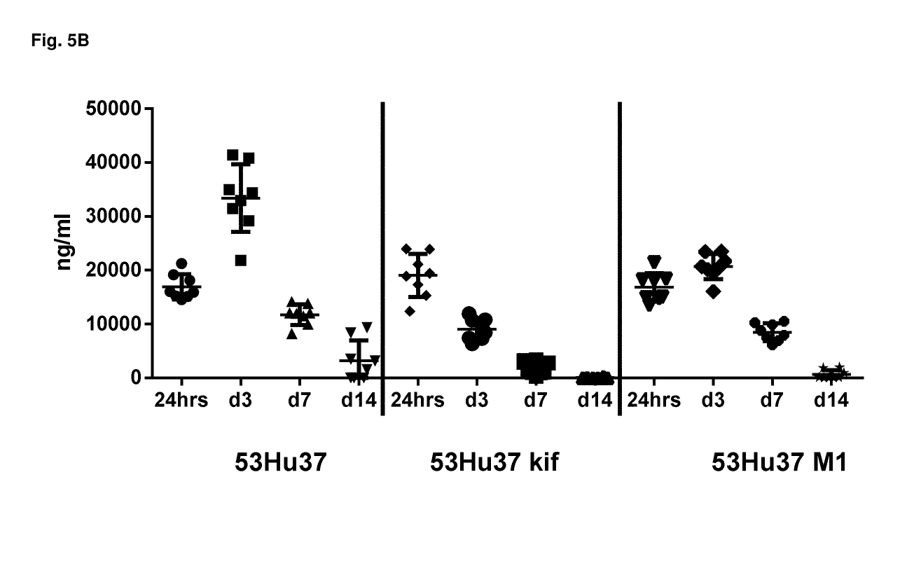

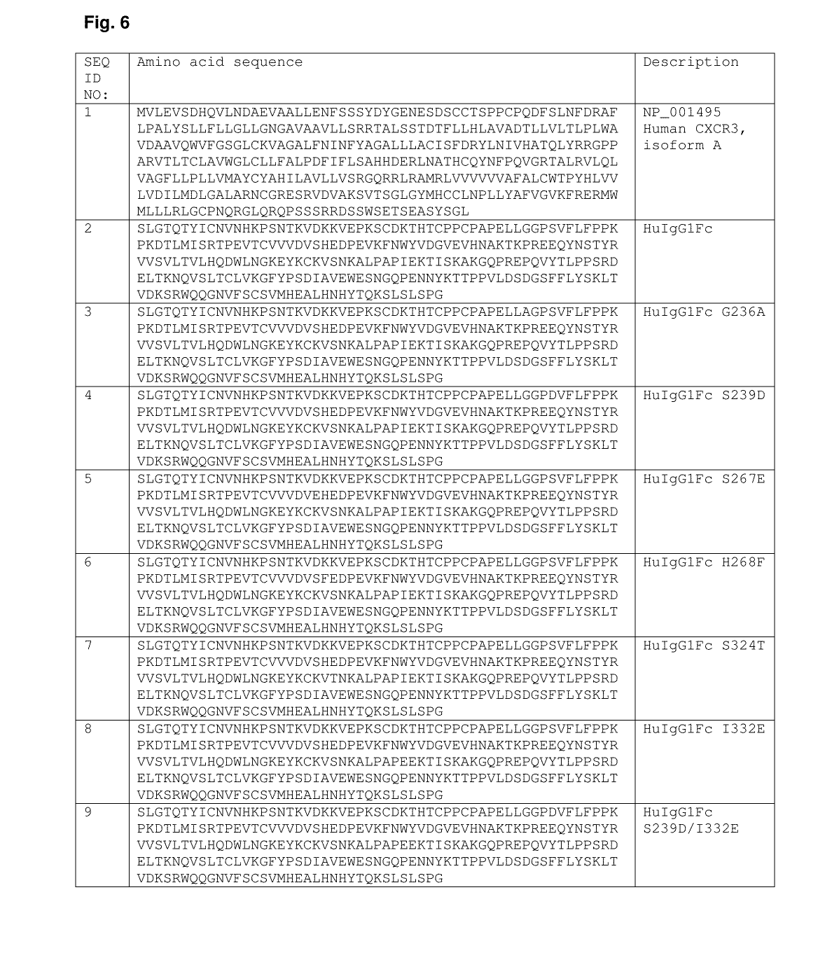

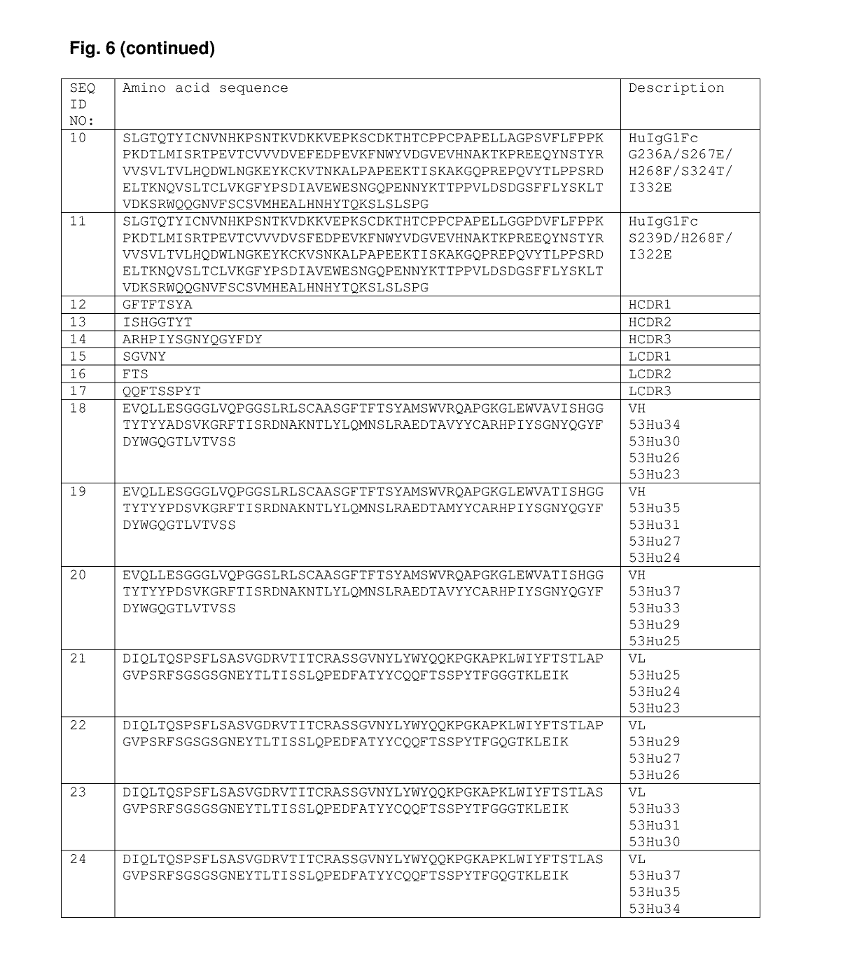

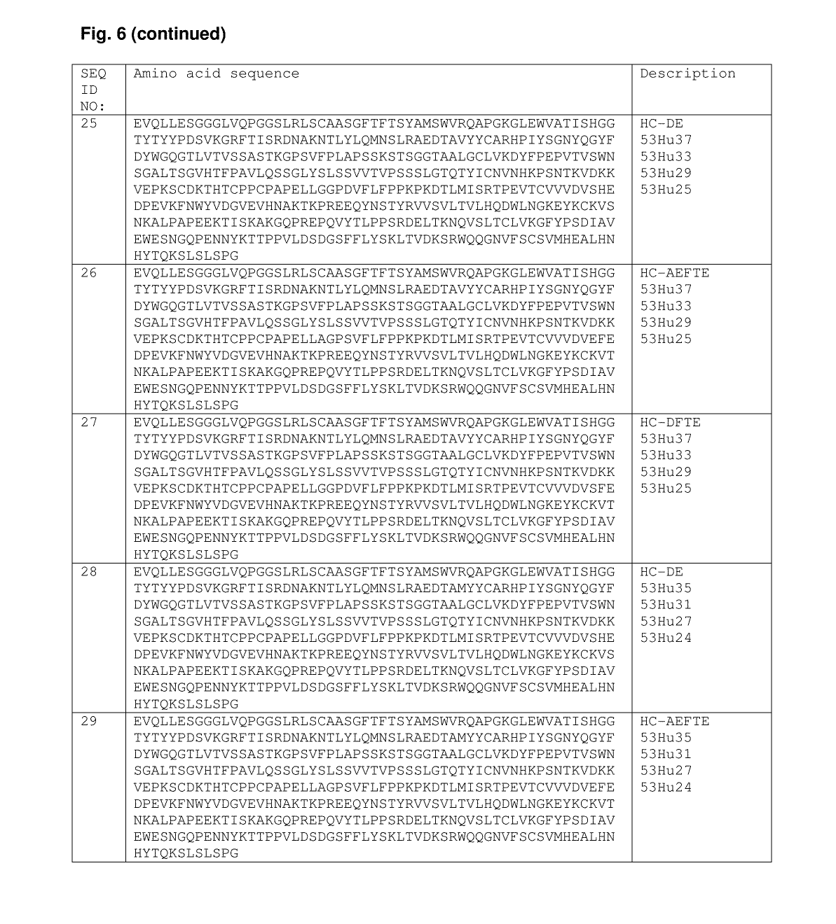

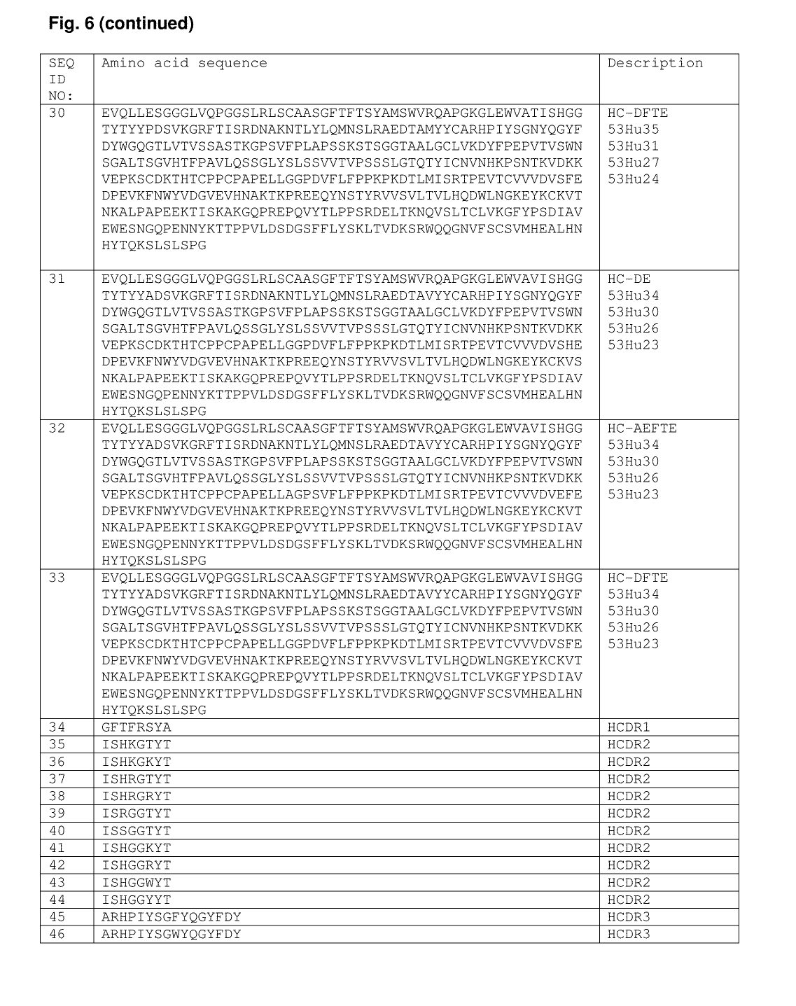

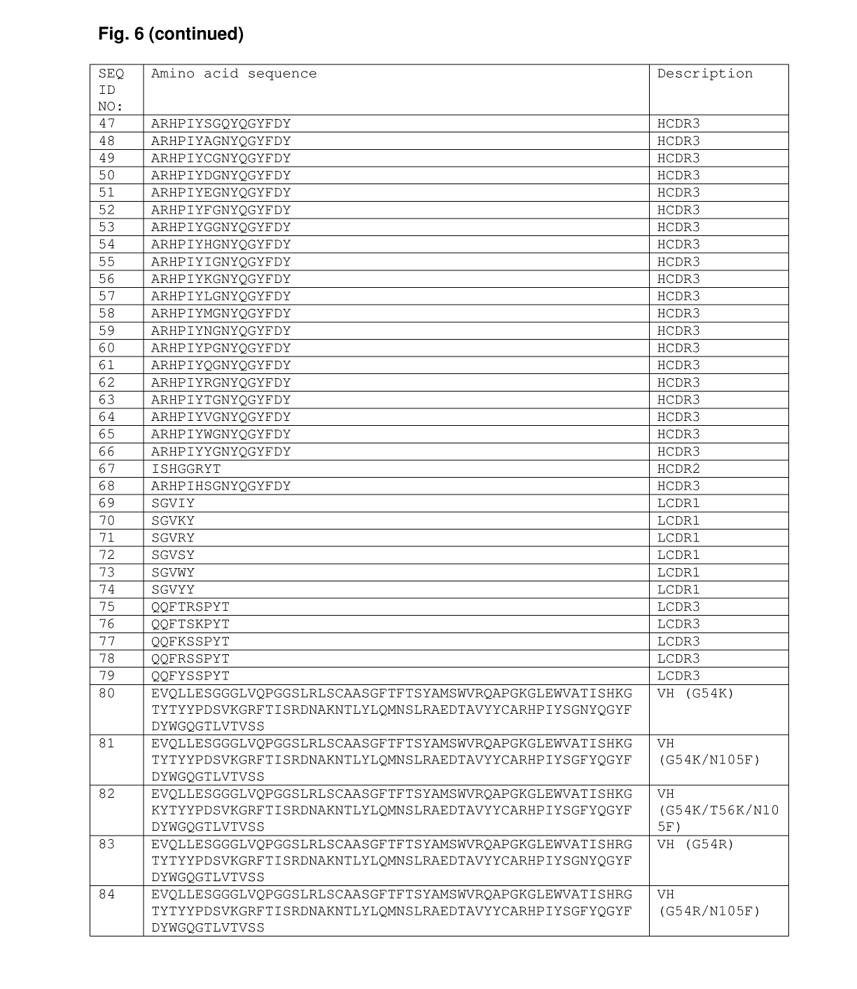

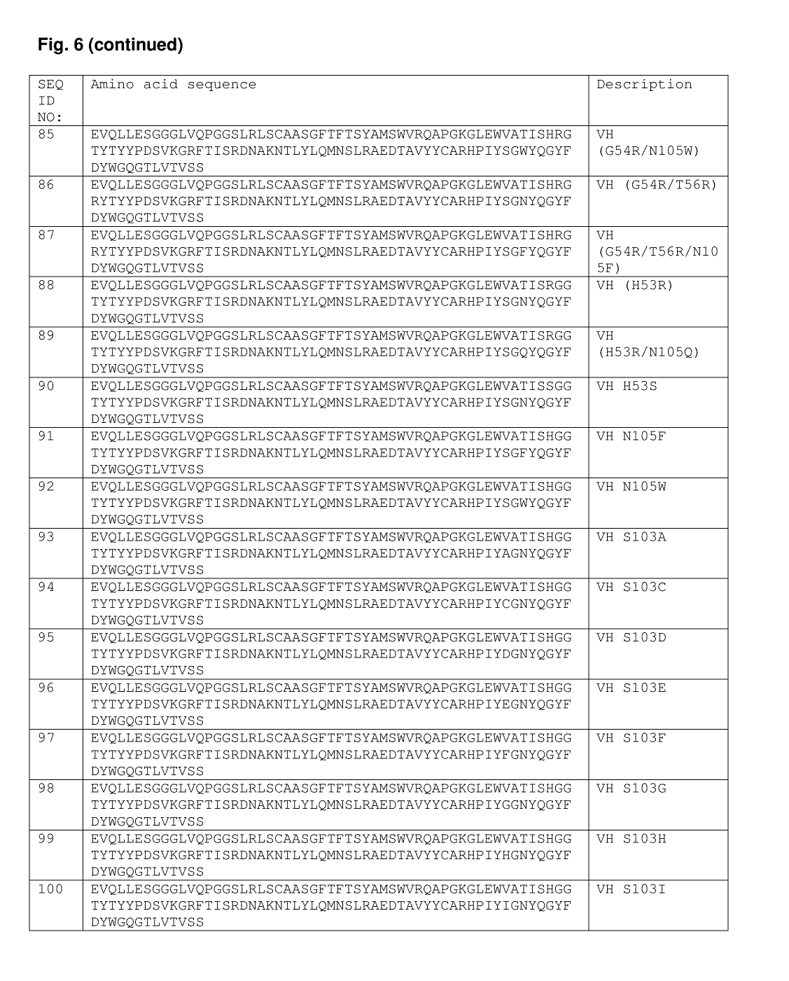

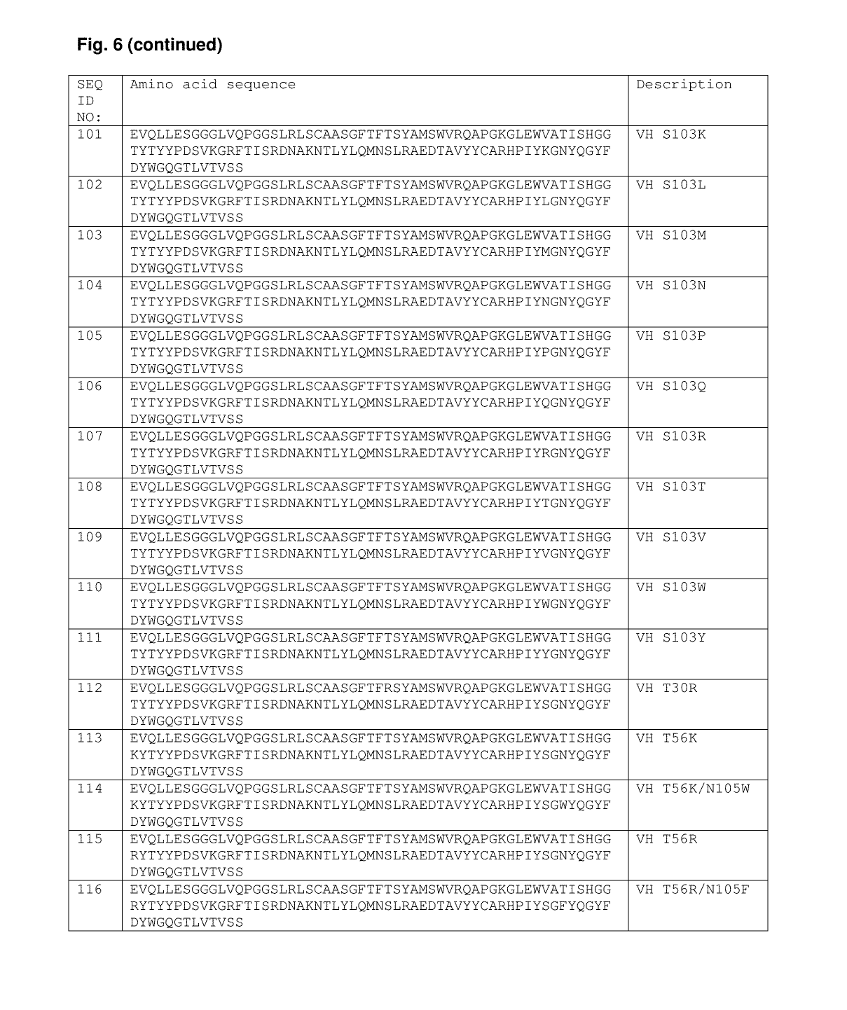

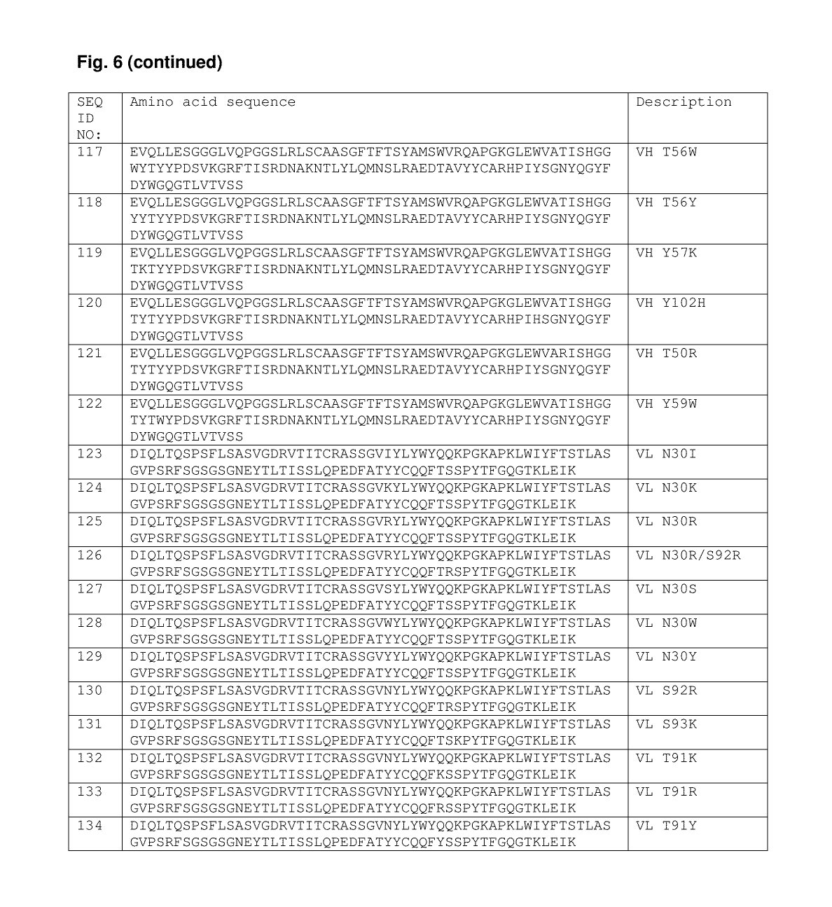

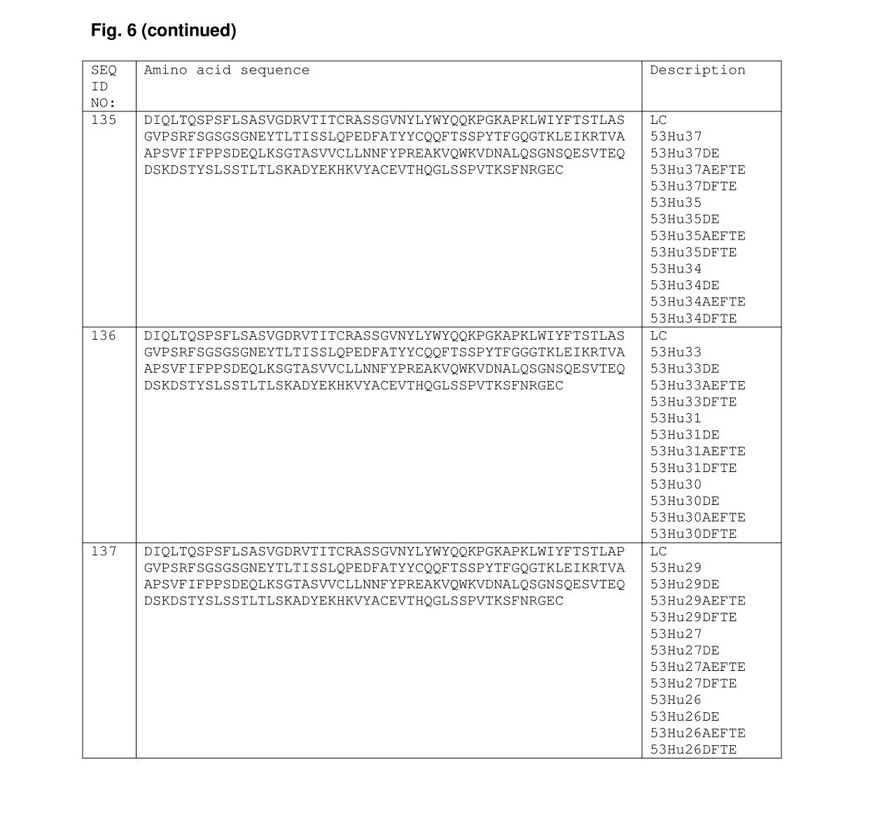

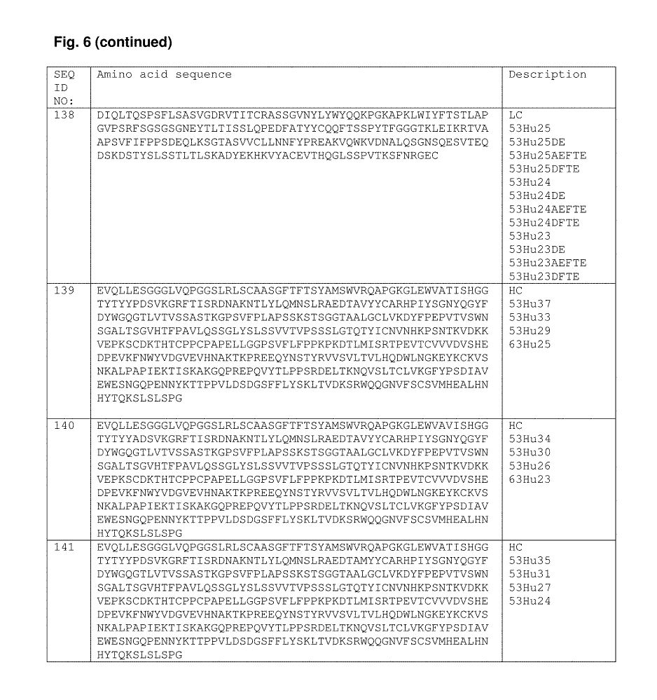

Cynomolgus monkeys received a single intravenous infusion of 2 mg/kg body weight of 53Hu37, the M1 variant, or kifunensine-treated version (n=8 per antibody treatment group), or vehicle control (n=6). Blood samples were collected before and then 1, 3, 7, and 14 days after infusion and analyzed by flow cytometry for total T cells and T-cell subsets. For flow cytometry, red blood cells were lysed and cells stained with antibodies for the following markers to identify total and memory CD4 and CD8 T cells: CD3 (clone SP34-2), CD4 (clone OKT4), CD8a (clone RPA-T8), CD45RA (clone 5H9), CCR7 (clone G043H7), and CXCR3 (clone 106). Cells were quantitated using Count Bright beads (Invitrogen) to determine cells/microliter (cells/μL). The data is represented as average percentage of the pre-bleed value for each cell subset for the group over time. Histology of spleen samples obtained from subsets of each treatment group at day 14 post infusion was studied by staining fixed sample sections for CXCR3 using the anti-human CXCR3 clone 4 antibody and the appropriate secondary antibody. Results are shown in FIGS. 5A-5C. Using peptide ELISA, sera from the blood samples were also assayed for pharmacokinetics, measuring circulating levels of administered antibody.

As shown in the figures, 53Hu37, the M1 version of 53Hu37, and Kif-treated 53Hu37 reduced the effector memory CD4 T cells and 53Hu37, the M1 version of Hu37, and Kif-treated 53Hu37 reduced the effector memory CD8 T cells. In addition, the M1 version of 53Hu37 and Kif-treated 53Hu37 dramatically reduced CXCR3 staining in spleens 14 days after single intravenous infusion of antibody.

### Example 8: Biochemical Analysis

Thermal stability, stability against shear stress and viral inactivation, stability against freeze-thaw stress, stability against agitation stress, and pH stability were measured for 53Hu37, the M1 version, and kifunensine-treated 53Hu37.

### Differential Scanning Calorimetry (DSC)

Samples were analyzed with a high throughput VP-DSC (Microcal). Samples were diluted to approximately 0.5 mg/mL with a corresponding buffer and were loaded onto a 96-well plate. The scan parameters consisted of a start temperature of 25° C. and an end temperature of 100° C. A scan rate of 200° C./h was used.

### Turbidity

Sample absorbance at 340-360 nm with 5 nm increments was measured on a Spectramax Plus (Molecular Devices) and the values averaged to yield the final turbidity measurement. 96-well UV flat bottom plates were used with 150-200 μL of material. The plates were pre-read before addition of sample and Path check was applied to the sample values. The samples were compared to values obtained from UV absorbance of turbidity standards based on “A Turbidimetric Method to Determine Visual Appearances of Protein Solutions” by Brigitte Eckhardt, Technology Applications Vol. 48, No. 2 March-April 1994 to classify the extent of turbidity.

### Size Exclusion Chromatography (SEC)

An HP1100 or 1200 series system was equipped with a TSK SW_{XL} size exclusion column coupled with a SW_{XL} guard column. Samples were run for 35 minutes using a mobile phase of 20 mM sodium phosphate, 500 mM NaCl, pH 6.0. A flow rate of 0.5 mL/min was used. Injections of 50 μg were performed and an UV signal was monitored at 280 nm.

### Thermal-Induced Relative Aggregation Propensity (TI-RAP)

Temperature-induced aggregation was produced by incubating 0.2 mg/mL anti-human CXCR3 antibodies (individual or mixture of antibodies) in PBS buffer and 10 mM histidine and 9% sucrose buffers at 5° C. (control), 64° C., 67° C., 70° C., or 73° C. for 10 min. After thermal incubation, samples were centrifuged at 7000×g for 2 minutes at 5° C. to remove insoluble protein precipitate and supernatants were analyzed by cation exchange chromatography (CEX). Percent of soluble monomer (and relative aggregation propensity) was calculated by normalizing chromatographic peak area for the thermally stressed samples using the peak area of a control (5° C.) sample.

### Agitation-Induced Relative Aggregation Propensity (AI-RAP)

For agitation-induced relative aggregation propensity, solution containing 0.2 mg/mL final protein concentration of anti-human CXCR3 antibodies in PBS buffer and 10 mM histidine and 9% sucrose buffers with 0 and 0.01% polysorbate 80 at 5° C. were subjected to rigorous agitation stress at 5° C. Solutions of anti-human CXCR3 antibodies were agitated at the highest speed from a VX-2500 Multi-Tube Vortexer (VWR, West Chester, Pa.) for a total duration of 24 h.

Various time points were analyzed throughout the study by removing small sample aliquots for CEX analysis. Sample aliquots were centrifuged at 7000×g (at 5° C.) for 2 minutes to remove insoluble protein precipitate and supernatants were and analyzed by CEX. Percent of soluble monomer (and relative aggregation propensity) was calculated by normalizing chromatographic peak area for the agitated samples using the peak area of a control (0 h) sample.

Cation Exchange Chromatography (CEX)

CEX analysis was performed on an Agilent 1290 infinity HPLC system using ProPac WCX-10 analytical column (weak cation exchange, 4×250 mm, Thermo Scientific) at 25° C. Twenty-microgram protein samples were loaded onto the column and analyzed at a flow rate of 0.8 mL/min. The column was equilibrated with Buffer A (20 mM sodium acetate, 0.0025% sodium azide, pH 5.2) and protein was eluted with a linear gradient of Buffer B (20 mM sodium acetate, 1 M sodium chloride, 0.0025% sodium azide, pH 5.2) from 0 to 100% over 40 minutes. Absorbance at 280 nm was measured and 280 nm absorbance peak was integrated to determine the protein peak area.

### Experimental Design

pH/Temperature Stress

A portion of the material from each clone was dialyzed into 20 mM sodium phosphate pH 5.0 and sodium phosphate pH 7.0 using Slide-A-Lyzers (Thermo Scientific PN 66810). This material was diluted using the corresponding buffer and filtered using Millex GV filters (Millipore PN SLGV033RB) to approximately 2 mg/mL. Samples in both sodium phosphate pH 5 and pH 7 were stored at 37° C. prior to testing at 3 and 5 weeks.

Freeze-Thaw Stress

The indicated anti-human CXCR3 antibodies were dialyzed into 20 mM sodium phosphate pH 6.0 using Slide-A-Lyzers (Thermo Scientific PN 66810). This material was diluted using the corresponding buffer and filtered using Millex GV filters (Millipore PN SLGV033RB) to approximately 1 mg/mL. Freeze-thaw stress samples were frozen at −80° C. and thawed at room temperature a total of 5 times. After 1 and 3 freeze-thaw cycles, material was removed for testing by select assays. A full analytic battery of testing was performed after the final freeze-thaw.

Shear Stress

Antibody samples were dialyzed into 20 mM sodium phosphate pH 6.0 using Slide-A-Lyzers (Thermo Scientific PN 66810). This material was diluted using the corresponding buffer and filtered using Millex GV filters (Millipore PN SLGV033RB) to approximately 1 mg/mL. Shear stress samples were repeatedly pipetted with a 200 μL pipette a total of 50 times.

Mock Viral Inactivation

Antibody samples were dialyzed into 20 mM sodium phosphate pH 6.0 using Slide-A-Lyzers (Thermo Scientific PN 66810). This material was diluted using the corresponding buffer and filtered using Millex GV filters (Millipore PN SLGV033RB) to approximately 1 mg/mL. Viral inactivation samples were brought to pH 3.5 with 1N HCl and held at that pH at room temperature for 100 minutes. Following the hold period, the samples were brought back to pH 7.2 using 1N NaOH and held for 100 minutes. Samples were then brought to pH 6.0 using 1 N HCl and tested.

### Results

Differential Scanning Calorimetry:

The M1 version (D/E mutant) was found to be slightly less stable than the Kif version, which in turn was slightly less stable than 53Hu37.

Shear Stress:

The M1 version (D/E mutant) was found to be slightly less stable than the 53Hu37 and the Kif version, the latter two being roughly equally stable.

Viral Inactivation Stress:

The M1 version (D/E mutant) was found to be more stable than the Kif version, both of which were less stable than 53Hu37.

Freeze-Thaw Stress:

The M1 version (D/E mutant), Kif version, and 53Hu37 were found to be roughly equivalent.

Agitation-Induced Relative Aggregation Propensity:

Approximately 80 percent of all versions of the antibody precipitated within 2 hours of agitation, although at pH 5.5, the M1 version (D/E mutant) was relatively more stable than the other two versions.

Aggregate Formation:

Under control (unaccelerated) conditions at pH 5.0, the M1 version (D/E mutant) and 53Hu37 were essentially stable over a 5-week period, but the Kif version became opalescent; this same pattern was found under accelerated conditions. Under control (unaccelerated) conditions at pH 7.0, the three versions of the antibody were roughly the same over a 5-week period. Under accelerated conditions the DE mutant was slightly more stable than 53Hu37, which in turn was more stable than the Kif version.

### Example 9: Depleting, but not Non-Depleting, Anti-CXCR3 is Protective and Therapeutic in a Mouse Model of Vitiligo

Mice.

KRT14-Kit1*4XTG2Bj1 (Krt14-Kit1*) mice were a gift from BJ Longley, University of Wisconsin. PMEL TCR transgenic mice are Thy1.1⁺, and were obtained from The Jackson Laboratory, stock no. 005023, B6.Cg Thy1^{a}/CyTg(TcraTcrb)8Rest/J. All mice were on a C57BL/6J background, maintained in pathogen-free facilities, and procedures were performed in accordance with the NIH Guide for the Care and Use of Laboratory Animals.

Antibodies.

deltaAB CXCR3 is an antibody that neutralizes CXCR3 activity (also referred to herein as IgG2a-dAB, dAB, or DAB). WT mouse CXCR3 is an antibody having a wild-type mouse IgG2a Fc that has depleting activity.

Vitiligo Induction & Antibody Treatments.

Vitiligo was induced through adoptive transfer of pre-melanosome specific (PMEL) CD8⁺ T cells as described previously. Harris et al., J. Invest. Dermatol. 132: 1869-76 (2012). Briefly, PMEL CD8⁺ T cells were isolated from the spleens of PMEL TCR transgenic mice (the Jackson Laboratory, stock no. 005023) through negative selection on microbeads (Miltenyi Biotech) according to the manufacturer's instructions. Purified CD8⁺ T cells (1×10⁶) were injected intravenously into sublethally irradiated (500 rads 1 day before transfer) Krt14-Kit1* hosts (the Jackson Laboratory stock no. 009687, 8-12 weeks of age). Recipient mice also received i.p. injection of 1×10⁶ pfu rVV-hPMEL (N Restifo, NCI, NIH) on the same day of transfer.

Treatment was performed by i.p. injection of 100 μg of antibody or isotype control three times weekly for the duration of the observation period.

For prevention studies, mice were treated from week 2-6 post-vitiligo induction. This time period is significant because it occurs after clearance of the virus used to induce disease, but before the onset of autoimmunity. Vitiligo score was quantified using a point scale based on the extent of depigmentation at four easily visible locations, including the ears, nose, rear footpads, and tails as previously described. Harris et al., J. Invest. Dermatol. 132: 1869-76 (2012). Each location was examined and the extent of depigmentation estimated as a percentage of the anatomic site; both left and right ears, and left and right rear footpads were estimated together and therefore evaluated as single sites. Points were awarded as follows: no evidence of depigmentation (0%) received a score of 0, >0-10%=1 point, >10-25%=2 points, >25-75%=3 points, >75−<100%=4 points, 100%=5 points. The “vitiligo score” was the sum of the scores at all four sites, with a maximum score of 20 points.

For repigmentation studies, mice with greater than 75% depigmentation on their tails were treated with WT mouse CXCR3 depleting Ab or isotype control from week 12-20 post-vitiligo induction, when disease is normally stable. Repigmentation analysis was performed with ImageJ as described previously. Agarwal et al., J. Invest. Dermatol. 135: 1080-8 (2015).

Effect of CXCR3 Antibodies on Cell Counts in Blood and Spleen.

Mice were infused with 100 μg of each antibody, and memory CD8⁺ T cell counts from blood (cells per microliter determined using Countbright beads) and spleen (total cell counts using a Via-Cell XR instrument from Becton Coulter) were assessed at 24 h. Because the dosed antibody is essentially the same antibody (same CDR) as the antibody used for CXCR3 staining and can block staining antibody from binding to CXCR3, depletion was assessed using a CXCR3-independent measure by evaluating the number of memory CD8⁺ T cells (as defined by CD44 and CD62L staining) since over 50% of these cells express CXCR3.

Flow Cytometry.

Blood, tails, spleens, and skin-draining lymph nodes were harvested and analyzed via flow cytometry. Spleens were disrupted, and the red blood cells lysed using RBC Lysis buffer (Sigma Aldrich). Blood samples were stained and treated with the Biolegend lysis/fixation buffer per the manufacturer's protocol. For separation of the dermis and epidermis, tail skin samples were incubated with 5 U mL⁻¹ dispase II (Roche) for 1 h at 37° C. Epidermis was removed and mechanically disrupted using 70 μm cell strainers, and dermis samples were incubated with 1 mg mL⁻¹ collagenase IV with 1-2 mg mL⁻¹ DNAse I (Sigma-Aldrich) for 1 h at 37° C. All cells were filtered through a 70 μm mesh prior to analysis. To identify PMELs and their phenotypes, samples were stained with the following antibodies at 1:200 in FACS buffer (1% BSA in PBS): CD45 AF700, CD8b PerCP-Cy5.5, Thy1.1 Pacific Blue, (Biolegend). Live/Dead Blue (Invitrogen) was used at 1:1000 in FACS buffer to distinguish live cells. The data were collected and analyzed using BD LSR II flow cytometer (BD Biosciences) and FlowJo Software Version 10 (Tree Star, Inc.).

Results. Effect of CXCR3 Antibodies on Cell Counts in Blood and Spleen.

In blood and spleen, the total number of memory CD8⁺ T cells was significantly reduced in mice treated with WT mouse or hamster CXCR3 antibody, whereas the number of cells was unchanged in mice treated with deltaAB CXCR3, compared to untreated mice.

Prevention.

Mice were treated with hamster anti-mouse CXCR3 (depleting), WT mouse CXCR3 (superior depleting), deltaAB CXCR3 (neutralizing; non-depleting), or isotype control antibody. Of these, the depleting antibody WT mouse CXCR3 performed the best in preventing clinical disease, as evidenced by significantly reduced scores 5 weeks post-treatment (one-way ANOVA p=0.0066, Dunnett's post-tests vs isotype ns; Tukey's post-tests significant for ΔAB vs WT and ΔAB vs hamster). This observation is consistent with data indicating that the WT mouse CXCR3 Ab has better depleting activity than the hamster CXCR3 Ab. All Abs tested in vitiligo prevention in mice resulted in fewer PMELs in the skin. PMEL numbers were significantly reduced in epidermis following treatment with any of the CXCR3 Abs (two-way ANOVA p<0.0001 with Bonferroni's post-tests compared to isotype control) and trended towards a reduction in the dermis. However, despite its ability to reduce PMEL number in the skin, neutralizing antibody was less effective than depleting antibodies, possibly due to the fact that a low threshold number of PMELs is sufficient for full clinical disease. The effect of CXCR3 depleting antibody on host T cells was assessed by measuring the number of host CD8⁺ T cells and total CD45⁺ cells in lymph nodes (LN), spleen, blood, epidermis, and dermis. Bystander host CD8⁺ T cell numbers were significantly reduced in the spleen and lymph nodes (LN) by hamster or WT mouse CXCR3 Ab treatment, but not in the skin (two-way ANOVA p<0.0001 with Bonferroni's post-tests compared to isotype control). However, total numbers of CD45⁺ cells were unchanged following treatment with any of the CXCR3 Abs.

Repigmentation.

Mouse WT CXCR3 Ab treatment significantly reversed clinical disease and reduced PMEL numbers in the epidermis. Host CD8⁺ T cell numbers were slightly reduced in treated mice, though total CD45⁺ cell numbers were unchanged. Taken together, these data indicate that CXCR3 depleting Ab can reduce autoreactive T cell numbers and reverse disease while having minimal impact on other compartments of the immune system.

The preceding examples are intended to illustrate and in no way limit the present disclosure. Other embodiments of the disclosed compounds and methods will be apparent to those skilled in the art from consideration of the specification and practice of the compounds and methods disclosed herein.

1.-15. (canceled)

16. A method of treating vitiligo, comprising administering to a subject in need thereof an effective amount of a humanized anti-human C-X-C motif chemokine receptor 3 (CXCR3) antibody comprising a heavy chain (HC) having a heavy chain variable region (VH) and light chain (LC) having a light chain variable region (VL), wherein
a) the VH and VL comprise amino acid sequences of sequence pairs selected from the group consisting of SEQ ID NOs:20/24, 18/22, 80/130, 81/24, 82/130, 82/24, 83/126, 83/130, 83/24, 84/126, 84/24, 85/24, 86/126, 87/126, 87/133, 87/24, 88/24, 89/24, 90/126, 91/126, 91/130, 92/130, 92/24, 93/126, 94/126, 95/126, 96/126, 97/126, 98/126, 99/126, 100/126, 101/126, 102/126, 103/126, 104/126, 105/126, 106/127, 108/127, 109/126, 110/127, 111/126, 112/126, 121/24, 113/130, 113/24, 114/130, 115/126, 115/130, 115/24, 116/126, 116/130, 116/24, 117/126, 118/126, 20/123, 20/124, 20/125, 20/126, 20/127, 20/128, 20/129, 20/130, 20/131, 20/132, 20/133, 20/134, 119/126, 122/126, and 120/130 respectively, and wherein
b) the heavy chain comprises a human IgG1 Fc region selected from the group consisting of SEQ ID NOs: 2, 3, 4, 5, 6, 7, 8, 9, 10, and 11.

17. The method of claim 16, wherein the VH comprises the amino acid sequence of SEQ ID NO:20 and the VL comprises the amino acid sequence of SEQ ID NO:24.

18. The method of claim 17, wherein the human IgG1 Fc region comprises the amino acid sequence of SEQ ID NO:2.

19. The method of claim 17, wherein the human IgG1 Fc region comprises the amino acid sequence of SEQ ID NO:9.

20. The method of claim 17, wherein the human IgG1 Fc region comprises the amino acid sequence of SEQ ID NO:10.

21. The method of claim 17, wherein the human IgG1 Fc region comprises the amino acid sequence of SEQ ID NO:11.

22. The method of claim 16, wherein the VH comprises the amino acid sequence of SEQ ID NO:18 and the VL comprises the amino acid sequence of SEQ ID NO:22.

23. The method of claim 22, wherein the human IgG1 Fc region comprises the amino acid sequence of SEQ ID NO:2.

24. The method of claim 22, wherein the human IgG1 Fc region comprises the amino acid sequence of SEQ ID NO:9.

25. The method of claim 22, wherein the human IgG1 Fc region comprises the amino acid sequence of SEQ ID NO:10.

26. The method of claim 22, wherein the human IgG1 Fc region comprises the amino acid sequence of SEQ ID NO:11.

27. The method of claim 16, wherein
a) the HC comprises the amino acid sequence of SEQ ID NO:25 and the LC comprises the amino acid sequence of SEQ ID NO: 135,
b) the HC comprises the amino acid sequence of SEQ ID NO:26, and the LC comprises the amino acid sequence of SEQ ID NO: 135,
c) the HC comprises the amino acid sequence of SEQ ID NO:27, and the LC comprises the amino acid sequence of SEQ ID NO:135,
d) the HC comprises the amino acid sequence of SEQ ID NO:139 and the LC comprises the amino acid sequence of SEQ ID NO:135,
e) the HC comprises the amino acid sequence of SEQ ID NO:31, and the LC comprises the amino acid sequence of SEQ ID NO:137,
f) the HC comprises the amino acid sequence of SEQ ID NO:32 and the LC comprises the amino acid sequence of SEQ ID NO:137,
g) the HC comprises the amino acid sequence of SEQ ID NO:33 and the LC comprises the amino acid sequence of SEQ ID NO:137, or
h) the HC comprises the amino acid sequence of SEQ ID NO:140 and the HC comprises the amino acid sequence of SEQ ID NO:137.

28. The method of claim 16, wherein the HC comprises a human IgG1 Fc region having reduced fucose content.

29. The method of claim 16, wherein the antibody is produced in a host cell that is cultured in the presence of a glycosylation inhibitor.

30. The method of claim 29, wherein the glycosylation inhibitor is kifunensine.

31. (canceled)

32. A kit for treating vitiligo, comprising
(a) a humanized anti-human CXCR3 antibody comprising a heavy chain (HC) having a heavy chain variable region (VH) and light chain (LC) having a light chain variable region (VL), wherein
(i) the VH and VL comprise amino acid sequences of sequence pairs selected from the group consisting of SEQ ID NOs:20/24, 18/22, 80/130, 81/24, 82/130, 82/24, 83/126, 83/130, 83/24, 84/126, 84/24, 85/24, 86/126, 87/126, 87/133, 87/24, 88/24, 89/24, 90/126, 91/126, 91/130, 92/130, 92/24, 93/126, 94/126, 95/126, 96/126, 97/126, 98/126, 99/126, 100/126, 101/126, 102/126, 103/126, 104/126, 105/126, 106/127, 108/127, 109/126, 110/127, 111/126, 112/126, 121/24, 113/130, 113/24, 114/130, 115/126, 115/130, 115/24, 116/126, 116/130, 116/24, 117/126, 118/126, 20/123, 20/124, 20/125, 20/126, 20/127, 20/128, 20/129, 20/130, 20/131, 20/132, 20/133, 20/134, 119/126, 122/126, and 120/130, respectively and wherein
(ii) the HC further comprises a human IgG1 Fc region comprising an amino acid sequence selected from the group consisting of SEQ ID NOs: 2, 3, 4, 5, 6, 7, 8, 9, 10, or 11; and
(b) instructions to administer the humanized anti-human CXCR3 to a human subject in need thereof.
